(19) United States
(12) Patent Application Publication
(10) Pub. No.: US 2022/0117874 A1
(43) Pub. Date: Apr. 21, 2022

(54) USE OF POLY-N-ACETYLGLUCOSAMINE NANOFIBERS FOR THE TREATMENT OF HAIR LOSS

(71) Applicant: Marine Polymer Technologies, Inc., Burtington, MA (US)

(72) Inventor: Sergio Finkielsztein, Newton, MA (US)

(73) Assignee: Marine Polymer Technologies, Inc., Burlington, MA (US)

(21) Appl. No.: 17/428,858

(22) PCT Filed: Feb. 7, 2020
(86) PCT No.: PCT/US2020/017185

§ 371 Date: Aug. 5, 2021

Related U.S. Application Data
(60) Provisional application No. 62/803,812, filed on Feb. 11, 2019.
(60) Provisional application No. 62/803,380, filed on Feb. 8, 2019.

(51) Int. Cl. A61K 8/73; A61K 8/02; A61Q 5/00; A61Q 7/00

(52) U.S. Cl. CPC A61K 8/73; A61K 8/027; B82Y 5/00; A61Q 7/00; A61Q 5/00

(57) ABSTRACT

In one aspect, described herein are methods for reducing hair shedding, comprising administering to the scalp, hair, or both of a subject a composition comprising shortened fibers of poly-N-acetylglucosamine. In another aspect, described herein are methods for improving hair growth, comprising administering to the scalp, hair, or both of a subject a composition comprising shortened fibers of poly-N-acetylglucosamine. In another aspect, described herein are methods for promoting healthier hair, comprising administering to the scalp, hair, or both of a subject a composition comprising shortened fibers of poly-N-acetylglucosamine. In another aspect, described herein are methods for improving hair growth and thickness of the diameter of hair fiber, comprising administering to the scalp, hair, or both of a subject a composition comprising shortened fibers of poly-N-acetylglucosamine.

This application claims priority to U.S. provisional application No. 62/803,380, filed on Feb. 8, 2019 and U.S. provisional application No. 62/803,812, filed on Feb. 11, 2019, each of which are incorporated herein by reference in their entirety.

### 1. FIELD

In one aspect, described herein are methods for reducing hair shedding, comprising administering to the scalp, hair, or both of a subject a composition comprising shortened fibers of poly-N-acetylglucosamine. In another aspect, described herein are methods for improving hair growth, comprising administering to the scalp, hair, or both of a subject a composition comprising shortened fibers of poly-N-acetylglucosamine. In another aspect, described herein are methods for promoting healthier hair, comprising administering to the scalp, hair, or both of a subject a composition comprising shortened fibers of poly-N-acetylglucosamine. In another aspect, described herein are methods for improving hair growth and thickness of the diameter of hair fiber, comprising administering to the scalp, hair, or both of a subject a composition comprising shortened fibers of poly-N-acetylglucosamine.

### 2. BACKGROUND

Hair is a physical structure of great cosmetic importance. Loss of hair is often a matter of concern in all individuals regardless of age and sex. In particular, hair is an essential part in identity for many women and men. Hair loss remains one of the most impactful quality of life disorders. Increased hair shedding, presenting in many dermatological and non-dermatological conditions, can have psychological impacts, such as, lowering self-esteem, anxiety and depression, (Tabolli et al. (2013) Health status, coping strategies, and alexithymia in subjects with androgenetic alopecia: A questionnaire study. American Journal of Clinical Dermatology, 14(2), 139-145). Health status, coping strategies, and alexithymia in subjects with androgenetic alopecia: A questionnaire study. American Journal of Clinical Dermatology, 14(2), 139-145). Typically, hair cycle results in replacement of every hair on the scalp by 3-5 years, (Habif TP. Clinical dermatology: A colour guide to diagnosis and therapy. 3rd edn. St. Louis: Mosby; 1996. Hair diseases. In: Habif T P, editor; pp. 739-47). Hair cycle is the sequential phases of growth and rest that each follicle goes through which includes the anagen phase (active hair growth), catagen phase (involution) and telogen phase (resting). In the normal scalp, 90-95% of the hair follicles are in the anagen phase and the remainder (5-10%) in the telogen phase with about 100-150 hair being shed daily. The molecular mechanisms underlying this phenomenon is complex and is still being unveiled. Numerous metabolic alterations such as pregnancy, malnutrition and other stressful conditions are capable of influencing the biological clock within hair follicles. Currently, no treatment exists and the standard of care includes mitigating stress and psychological counseling. Accordingly, there is a need for understanding the prevalence of hair shedding and treatments for addressing hair shedding.

### 3. SUMMARY

Described herein are methods for improving hair growth, promoting healthy hair, reducing hair shedding, increasing the diameter of hair fiber, promoting a healther scalp, and strengthening hair follicles using sNAG nanofibers. In some aspects, provided herein are methods for reducing hair shedding, the method comprising applying a composition comprising shortened fibers of poly-N-acetylglucosamine (“sNAG nanofibers”) to the scalp or the scalp and hair of a subject and leaving the composition on the scalp or the scalp and hair for a period of time (e.g., at least 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours), wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides. In certain embodiments, the composition is applied to the scalp or the scalp and hair and it is not removed until the subject washes his/her hair the next time. In some embodiments, the subject washes his/her hair 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours after the composition has been applied to their scalp or their scalp and hair.

In some aspects, provided herein are methods for improving hair growth, the method comprising applying a composition comprising shortened fibers of poly-N-acetylglucosamine (“sNAG nanofibers”) to the scalp or the scalp and hair of a subject and leaving the composition on the scalp or the scalp and hair for a period of time (e.g., at least 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours), wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides. In certain embodiments, the composition is applied to the scalp or the scalp and hair and it is not removed until the subject washes his/her hair the next time. In some embodiments, the subject washes his/her hair 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours after the composition has been applied to their scalp or their scalp and hair.

In some aspects, provided herein are methods for promoting healthier hair, the method comprising applying a composition comprising shortened fibers of poly-N-acetylglucosamine (“sNAG nanofibers”) to the scalp or the scalp and hair of a subject and leaving the composition on the scalp or the scalp and hair for a period of time (e.g., at least 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours), wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides. In some embodiments, the healthier hair promoted has one, two, or more, or all of the following characteristics: thicker hair, hair with improved texture, hair with greater volume, and softer hair. In certain embodiments, the composition is applied to the scalp or the scalp and hair and it is not removed until the subject washes his/her hair the next time. In some embodiments, the subject washes his/her hair 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours after the composition has been applied to their scalp or their scalp and hair.

In some aspects, provided herein are methods for improving the health a subject's scalp, the method comprising applying a composition comprising shortened fibers of poly-N-acetylglucosamine (“sNAG nanofibers”) to the scalp or the scalp and hair of the subject and leaving the composition on the scalp or the scalp and hair for a period of time (e.g., at least 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours), wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides. In certain embodiments, the composition is applied to the scalp or the scalp and hair and it is not removed until the subject washes his/her hair the next time. In some embodiments, the subject washes his/her hair 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours after the composition has been applied to their scalp or their scalp and hair.

In some aspects, provided herein are methods for strengthening hair follicles, the method comprising applying a composition comprising shortened fibers of poly-N-acetylglucosamine (“sNAG nanofibers”) to the scalp or the scalp and hair of a subject and leaving the composition on the scalp or the scalp and hair for a period of time (e.g., at least 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours), wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides. In certain embodiments, the composition is applied to the scalp or the scalp and hair and it is not removed until the subject washes his/her hair the next time. In some embodiments, the subject washes his/her hair 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours after the composition has been applied to their scalp or their scalp and hair.

In some aspects, provided herein are methods for thickening the diameter of existing hair fiber, the method comprising applying a composition comprising shortened fibers of poly-N-acetylglucosamine (“sNAG nanofibers”) to the scalp or the scalp and hair of a subject and leaving the composition on the scalp or the scalp and hair for a period of time (e.g., at least 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours), wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides. In certain embodiments, the composition is applied to the scalp or the scalp and hair and it is not removed until the subject washes his/her hair the next time. In some embodiments, the subject washes his/her hair 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours after the composition has been applied to their scalp or their scalp and hair.

In some aspects, provided herein are methods for improving hair growth and thickening the diameter of existing hair fiber, the method comprising applying a composition comprising shortened fibers of poly-N-acetylglucosamine (“sNAG nanofibers”) to the scalp or the scalp and hair of a subject and leaving the composition on the scalp or the scalp and hair for a period of time (e.g., at least 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours), wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides. In certain embodiments, the composition is applied to the scalp or the scalp and hair and it is not removed until the subject washes his/her hair the next time. In some embodiments, the subject washes his/her hair 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours after the composition has been applied to their scalp or their scalp and hair.

In some aspects, provided herein are methods for treating hair loss, the method comprising applying a composition comprising shortened fibers of poly-N-acetylglucosamine (“sNAG nanofibers”) to the scalp or the scalp and hair of a subject and leaving the composition on the scalp and hair for a period of time (e.g., at least 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours), wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides. In certain embodiments, the composition is applied to the scalp or the scalp and hair and it is not removed until the subject washes his/her hair the next time. In some embodiments, the subject washes his/her hair 1 hour, 2 hours 3 hours, 6 hours, 9 hours, 12 hours, 18 hours, or 24 hours, or 1-3 hours, 3-6 hours, 6-9 hours, 6-12 hours, 9-12 hours, 12-18 hours, 12-24 hours, or 18-24 hours after the composition has been applied to their scalp or their scalp and hair.

In some embodiments, a composition described herein is applied to the scalp of a subject after shampooing, conditioning, or shampooing and conditioning the hair. In a specific embodiment, a composition described herein is sprayed directly on the scalp or as close to the scalp as possible and the composition is massaged in. For example, a subject's hair may be parted several times in order to spray a composition described herein directly on the entire scalp or as close to the entire scalp as possible and the composition is massaged in. In some embodiments, a composition described herein is applied to a dry scalp. In some embodiments, a composition described herein is applied to wet hair. In some embodiments, a composition described herein is applied once daily. In some embodiments, a composition described herein is applied once daily for a period of approximately 1 to approximately 3 months. In certain embodiments, a composition described herein is applied once daily for approximately 3 to approximately 6 months. In some embodiments, a composition described herein is applied once daily for approximately 6 to approximately 12 months. In certain embodiments, a composition described herein is applied twice daily for approximately 3 to approximately 6 months. In some embodiments, a composition described herein is massaged into the scalp and hair.

In a specific embodiment, a composition described herein comprises water (e.g., purified water). In another specific embodiment, a composition described herein comprises sNAG nanofibers at a concentration of approximately 1 mg/mL. In another specific embodiment, a composition described herein comprises sNAG nanofibers suspended in water (e.g., purified water) at a concentration of 0.05 mg/mL to 5 mg/mL (in some embodiments, 1 mg/mL). In some embodiments, a composition described herein comprises phenoxyethanol (e.g., 0.6% phenoxyethanol), caprylyl glycol, and sodium hydroxide in addition to sNAG nanofibers. In certain embodiments, a composition described herein comprises glucosamine (e.g., D(+)glucosamine). In some embodiments, a composition described herein comprises sNAG nanofibers and does not comprise an additional ingredient.

In specific embodiments, approximately 75 microliters to approximately 200 microliters of a composition described herein is sprayed multiple times (e.g., 5 to 30, 5 to 25, 5 to 20, 5 to 15, or 5 to 10 times) over the entire scalp or the entire scalp and hair, and the composition is massaged into the scalp or the hair and scalp for a period of time (e.g., for approximately 10 to 15 seconds, approximately 10 to 30 seconds, approximately 30 to 60 seconds, approximately 30 to 90 seconds, approximately 1 to 3 minutes, or approximately 1 to 5 minutes). In certain embodiments, 0.5 to 3 mg, 0.5 to 2 mg, 0.5 to 1 mg, 1 to 3 mg, 2 to 3 mg, 0.8 to 1 mg, 0.8 to 2 mg, or 0.8 to 1 mg of sNAG nanofibers are applied to the scalp, hair or both of a subject daily for a period of time (e.g., for about 60 days, about 90 days, about 4 months, about 5 months, about 6 months, about 7 months, about 8 months, about 9 months or longer).

In some embodiments, a composition described herein is not administered in conjunction with another therapy. In specific embodiments, a composition described herein is not administered in conjunction with a therapy for the scalp or the hair or scalp (e.g., a therapy for improving hair health, a therapy for reducing hair loss, a therapy for the thickness of existing hair fiber, etc.).

As demonstrated in the examples below (see Section 5), the reduction of hair shedding is superior with sNAG nanofibers than with chitosan. In addition, as demonstrated in the example section below (see Section 5.4) the reduction of hair shedding is better with glucosamine than with chitosan. Further, as demonstrated by the examples below (see Section 5), a sNAG nanofiber composition reduced hair shedding more than either glucosamine or chitosan.

The subject to be treated in accordance with the methods described herein may be a mammal, preferably a human. In a preferred embodiment, the subject is a human subject. In a specific embodiment, the human subject is a human adult. In another specific embodiment, the human subject is an elderly human. In certain embodiments, the subject treated in accordance with the methods described herein does not have a viral infection of the scalp or a condition affecting the scalp caused by a viral infection. In some embodiments, the subject treated in accordance with the methods described herein does not have a bacterial infection of the scalp or a condition affecting the scalp caused by a bacterial infection. In certain embodiments, the subject treated in accordance with the methods described herein does not have a fungal infection of the scalp or a condition affecting the scalp caused by a fungal infection. In some embodiments, the subject does not have psoriasis (e.g. psoriasis affecting the scalp). In some embodiments, the subject treated in accordance with the methods described herein does not have dermatitis (e.g. dermatitis affecting the scalp). In certain embodiments, the subject treated in accordance with the methods described herein does not have a wound on their scalp. In some embodiments, the subject treated in accordance with the methods described herein does not have any form of baldness or alopecia.

The sNAG nanofibers contemplated in the methods described herein may be of varying lengths, widths and molecular weights as described in Section 4.1, infra. In certain embodiments, the majority (and in certain embodiments, at least or more than 60%, 70%, 80%, 90%, 95% or 99%) of the sNAG nanofibers, or 100% of the sNAG nanofibers, are between about 1 to 15 μm in length. In some embodiments, the majority (and in certain embodiments, at least or more than 60%, 70%, 80%, 90%, 95% or 99%) of the sNAG nanofibers, or 100% of the sNAG nanofibers, are between about 2 to 10 μm, or 4 to 7 μm in length. In certain embodiments, more than 50% of the sNAG nanofibers in a composition described herein are between about 2 to 10 μm in length. In a specific embodiment, 100% of the sNAG nanofibers in a composition described herein are between about 1 to 15 μm in length. In a particular, embodiment, the length of the sNAG nanofibers is determined by scanning electron microscopy. The sNAG nanofibers of the described length can be obtained, for example, as described below in Section 4.2, infra. In a specific embodiment, the poly-N-acetylglucosamine nanofibers have a β-1→4 configuration.

In certain embodiments, the sNAG nanofibers in a composition described herein have an average length of between about 1 to about 10 μm in length. In some embodiments, the sNAG nanofibers in a composition described herein have an average length of between about 4 to about 7 μm in length. In certain embodiments, the sNAG nanofibers in a composition described herein have an average length of between about 1 to about 5 μm in length. In a particular embodiment, the length of the sNAG nanofibers is determined by scanning electron microscopy. In a specific embodiment, the poly-N-acetylglucosamine nanofibers have a β-1→4 configuration.

In certain embodiments, the sNAG nanofibers described herein have a molecular weight of approximately 60,000 to approximately 80,000 daltons. In a specific embodiment, the sNAG nanofibers described herein have a molecular weight of approximately 60,000 daltons, approximately 65,000 daltons, approximately 70,000 daltons, approximately 75,000 daltons or approximately 80,000 daltons.

In certain embodiments, the sNAG nanofibers in a composition described herein have an average length of between about 1 to 10 μm in length and molecular weight of approximately 50,000 daltons to approximately 100,000 daltons. In certain embodiments, the sNAG nanofibers in a composition described herein have an average length of between about 1 to 10 μm in length and molecular weight of approximately 60,000 daltons to approximately 80,000 daltons. In some embodiments, the sNAG nanofibers in a composition described herein have an average length of between about 1 to 10 μm in length and molecular weight of approximately 50,000 daltons, approximately 55,000 daltons, approximately 60,000 daltons, approximately 65,000 daltons, approximately 70,000 daltons, approximately 75,000 daltons, approximately 80,000 daltons, approximately 85,000 daltons, approximately 90,000 daltons, approximately 95,000 daltons, or approximately 100,000 daltons. In a particular embodiment, the length of the sNAG nanofibers is determined by scanning electron microscopy. In a specific embodiment, the poly-N-acetylglucosamine nanofibers have a β-1→4 configuration.

In certain embodiments, the sNAG nanofibers in a composition described herein have an average length of between about 1 to 8 μm in length and molecular weight of approximately 50,000 daltons to approximately 100,000 daltons. In some embodiments, the sNAG nanofibers in a composition described herein have an average length of between about 4 to 7 μm in length and molecular weight of approximately 50,000 daltons, approximately 55,000 daltons, approximately 60,000 daltons, approximately 65,000 daltons, approximately 70,000 daltons, approximately 75,000 daltons approximately 80,000 daltons, approximately 85,000 daltons, approximately 90,000 daltons, approximately 95,000 daltons, or approximately 100,000 daltons. In some embodiments, the sNAG nanofibers in a composition described herein have an average length of between about 1 to 8 μm in length and molecular weight of approximately 50,000 daltons, approximately 55,000 daltons, approximately 60,000 daltons, approximately 65,000 daltons, approximately 70,000 daltons, approximately 75,000 daltons approximately 80,000 daltons, approximately 85,000 daltons, approximately 90,000 daltons, approximately 95,000 daltons, or approximately 100,000 daltons. In certain embodiments, the sNAG nanofibers in a composition described herein have an average length of between about 1 to about 8 μm in length or about 1 to 5 μm in length and molecular weight of 60,000 daltons to 80,000 daltons. In some embodiments, the sNAG nanofibers in a composition described herein have an average length of between about 1 to about 8 μm in length or about 1 to 5 μm in length and molecular weight of 70,000 daltons±6,000 daltons. In a particular embodiment, the length of the sNAG nanofibers is determined by scanning electron microscopy. In a specific embodiment, the poly-N-acetylglucosamine nanofibers have a β-1→4 configuration.

In a specific embodiment, the sNAG nanofibers comprise glucosamine monosaccharides, wherein at least 70% of the monosaccharides are N-acetylglucosamine monosaccharides. In another embodiment, the sNAG nanofibers comprise glucosamine monosaccharides, wherein at least 90% of the monosaccharides are N-acetylglucosamine monosaccharides. In another embodiment, the sNAG nanofibers comprise glucosamine monosaccharides, wherein at least 85% of the monosaccharides are N-acetylglucosamine monosaccharides. In another embodiment, the sNAG nanofibers comprise glucosamine monosaccharides, wherein at least 95% of the monosaccharides are N-acetylglucosamine monosaccharides. In another embodiment, the sNAG nanofibers comprise glucosamine monosaccharides, wherein 70% to 80%, 70% to 95%, 75% to 80%, 75% to 90%, 75% to 95%, 80% to 90%, 85% to 90%, or 80% to 95% of the monosaccharides are N-acetylglucosamine monosaccharides.

In certain embodiments, the sNAG nanofibers in a composition described herein have an average length of between about 1 to 5 μm in length and molecular weight of approximately 50,000 daltons to approximately 100,000 daltons. In some embodiments, the sNAG nanofibers in a composition described herein have an average length of between about 1 to 5 μm in length and molecular weight of and molecular weight of approximately 50,000 daltons, approximately 55,000 daltons, approximately 60,000 daltons, approximately 65,000 daltons, approximately 70,000 daltons, approximately 75,000 daltons, approximately 80,000 daltons, approximately 85,000 daltons, approximately 90,000 daltons, approximately 95,000 daltons, or approximately 100,000 daltons. In a particular embodiment, the length of the sNAG nanofibers is determined by scanning electron microscopy. In a specific embodiment, the poly-N-acetylglucosamine nanofibers have a β-1→4 configuration.

In certain embodiments, the sNAG nanofibers in a composition described herein have an average length of between about 1 to about 8 μm in length or about 1 to 5 μm in length and have molecular weight of 60,000 daltons to 80,000 daltons, wherein the sNAG nanofibers comprises glucosamine monosaccharides and wherein at least 70% of the glucosamine monosaccharides are N-acetylglucosamine monosaccharides. In some embodiments, the sNAG nanofibers in a composition described herein have an average length of between about 1 to about 8 μm in length or about 1 to 5 μm in length and molecular weight of 70,000 daltons±6,000 daltons, wherein the sNAG nanofibers comprises glucosamine monosaccharides and wherein at least 70% of the glucosamine monosaccharides are N-acetylglucosamine monosaccharides. In a particular embodiment, the length of the sNAG nanofibers is determined by scanning electron microscopy. In a specific embodiment, the poly-N-acetylglucosamine nanofibers have a β-1→4 configuration.

In certain embodiments, the sNAG nanofibers in a composition described herein were produced by gamma irradiation of poly-N-acetylglucosamine fibers, and wherein the poly-N-acetylglucosamine fibers were irradiated in the form of dried fibers at 500-2,000 kgy, or the poly-β-N-acetylglucosamine fibers were irradiated in the form of wet fibers at 100-500 kgy. In a specific embodiment, the sNAG nanofibers in a composition described herein were produced from a microalgae poly-N-acetylglucosamine. In certain embodiments, the sNAG nanofibers in a composition described herein were produced by gamma irradiation of poly-N-acetylglucosamine fibers, and wherein the poly-N-acetylglucosamine fibers were irradiated in the form of wet fibers at 300 kgy.

In certain embodiments, the sNAG nanofibers are derived from microalgae. In another embodiment, the sNAG nanofibers are not derived from crustaceans. In yet another embodiment, the sNAG nanofibers may be derived from microalgae, crustaceans (e.g., shrimp), fungus or any other source.

In certain embodiments, the sNAG nanofibers used in the methods described herein are non-reactive in a biocompatibility test or tests. For example, the sNAG nanofibers used in the methods described herein may be non-reactive when tested in an elution test, an intramuscular implantation test, an intracutaneous test, or a systemic test. In some embodiments, the compositions described herein are non-reactive when tested in an elution test, an intramuscular implantation test, an intracutaneous test, or a systemic test. In other embodiments, the sNAG nanofibers used in the methods described herein have Grade 0 or Grade 1 when tested in an elution test, an intramuscular implantation test, an intracutaneous test, or a systemic test. In yet another embodiment, the sNAG nanofibers used in the methods described herein are at most mildly reactive when tested in an elution test, an intramuscular implantation test, an intracutaneous test, or a systemic test. In one embodiment, the sNAG nanofibers or compositions comprising such nanofibers are non-reactive as determined by an intramuscular implantation test. In certain embodiments, the compositions described herein do not cause an allergenic reaction or an irritation, e.g., at the site of application. In other embodiments, the compositions described herein cause at most a mild allergenic reaction or a mild irritation, e.g., at the site of application.

### 3.1 Terminology

As used herein, the terms “about” and “approximately” mean a range around a given value wherein the resulting value is the same or substantially the same (e.g., within 10%, 5% or 1%) as the expressly recited value. In one embodiment, “about” means within 10% of a given value or range. In another embodiment, the term “about” means within 5% of a given value or range. In another embodiment, the term “about” means within 1% of a given value or range.

As used herein, the term “elderly human” refers to a human 65 years or older.

As used herein, the term “human adult” refers to a human that is 18 years or older.

As used herein, the term “human child” refers to a human that is 1 year to 18 years old.

As used herein, the term “majority” refers to greater than 50%, including, e.g., 50.5%, 51%, 55%, etc.

As used herein, the term “subject” and “patient” are used interchangeably to refer to an animal (e.g., cow, horse, sheep, pig, chicken, turkey, cat, dog, mouse, rat, rabbit, guinea pig, etc.). In a specific embodiment, the subject is a mammal such as a non-primate or a primate, e.g., a human. In specific embodiments, the subject is a human. See Section 4.5, infra, for more information concerning patients treated in accordance with the methods provided herein.

### 4. DETAILED DESCRIPTION

Described herein are methods for treating hair loss conditions, improving hair health, reducing hair loss, improving hair growth (e.g., the rate of hair growth), improving the thickness of the diameter of existing hair fiber, and improving the condition of the scalp, comprising topically administering to the scalp, hair or both a subject a composition comprising shortened fibers of poly-N-acetylglucosamine or a derivative thereof.

4.1 sNAG Nanofibers

Described herein are shortened fibers of poly-N-acetylglucosamine (“sNAG” nanofibers). The sNAG nanofibers comprise fibers of poly-N-acetylglucosamine or a derivative(s) thereof, the majority of which are less than 30 microns in length and at least 1 micron in length as determined by any method known to one skilled in the art, for example, by scanning electron microscopy (“SEM”). In a specific embodiment, the sNAG nanofibers comprise fibers of poly-N-acetylglucosamine and not a derivative thereof, the majority of which are less than 30 microns in length and at least 1 micron in length as determined by any method known to one skilled in the art, for example, by scanning electron microscopy (“SEM”). Such sNAG nanofibers may be obtained, for example, as described herein. See, e.g., Section 4.2, infra for methods of making sNAG nanofibers. In a specific embodiment, a sNAG nanofiber is as described in Section 5, infra (in particular Sections 5.1 to 5.3, 5.5, and 5.6, infra).

In certain embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers are less than about 30, 25, 20, 15, 12, 10, 9, 8, 7, 6, 5, 4, or 3 microns in length, and at least 1 micron in length as determined by any method known to one skilled in the art, for example, by SEM. In specific embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers are less than about 20 microns, less than about 15 microns or less than about 10 microns in length, and at least 1 micron in length as determined by any method known to one skilled in the art, for example, by SEM. In another embodiment, at least 75%, at least 80%, at least 90%, or at least 95% of the sNAG nanofibers are less than about 15 microns in length, and at least 1 micron in length as determined by any method known to one skilled in the art, for example, SEM. In specific embodiments, all (100%) of the sNAG nanofibers are between about 1 micron and about 15 microns in length as determined by any method known to one skilled in the art, for example, by SEM. In certain embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers are equal to or less than 15, 14, 13, 12, 11, 10, 9, 8 or 7 microns in length, and at least 1 micron in length as determined by any method known to one skilled in the art, for example, by SEM. In some embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers are between 1 to 15, 2 to 15, 3 to 15, 2 to 14, 1 to 12, 2 to 12, 1 to 10, 2 to 10, 3 to 12, 3 to 10, 1 to 9, 2 to 9, 3 to 9, 1 to 8, 2 to 8, 3 to 8, 4 to 8, 1 to 7, 2 to 7, 3 to 7, 4 to 7, 1 to 6, 1 to 5, 1 to 4, or 1 to 3 microns in length as determined by any method known to one skilled in the art, for example, by SEM.

In a specific embodiment, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers are about 8, 7, 6, 5, 4, 3 or 2 microns in length as determined by any method known to one skilled in the art, for example, by SEM. In another specific embodiment, the majority of the sNAG nanofibers are about 8 microns in length as determined by any method known to one skilled in the art, for example, SEM. In another specific embodiment, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers are between about 2 to about 10 microns, about 1 to about 10 microns, about 1 to about 8 microns, about 3 to about 8 microns, or about 4 to about 7 microns in length as determined by any method known to one skilled in the art, for example, by SEM. In another specific embodiment, all (100%) of the sNAG nanofibers are between about 1 to about 15 microns, about 3 to about 15 microns, about 2 to about 10 microns, about 1 to about 10 microns, about 1 to about 8 microns, about 3 to about 8 microns, or about 4 to about 7 microns in length as determined by any method known to one skilled in the art, for example, by SEM. In another specific embodiment, all (100%) of the sNAG nanofibers are between about 1 to about 15 microns in length as determined by any method known by one skilled in the art, for example, by SEM.

In another embodiment, the average length of the sNAG nanofibers is about 15, 14, 13, 12, 11, 10, 9, 8, 7, 6, 5, 4, 3 or 2 microns as determined by any method known to one skilled in the art, for example, by SEM. In another specific embodiment, the average length of the sNAG nanofibers is between about 2 to about 10 microns, about 3 to about 8 microns, or about 4 to about 7 microns as determined by any method known to one skilled in the art, for example, by SEM. In another specific embodiment, the average length of the sNAG nanofibers is between about 1 to about 15 microns, about 3 to about 15 microns, about 2 to about 10 microns, about 3 to about 8 microns, about 4 to about 7 microns, or about 2 to about 10 microns, as determined by any method known to one skilled in the art, for example, by SEM. In a specific embodiment, the average length of the sNAG nanofibers is about 1 to 15 microns as determined by any method known to one skilled in the art, for example, by SEM. In a specific embodiment, the average length of the sNAG nanofibers is about 1 to about 8 microns or about 1 to about 5 microns.

In certain embodiments, the sNAG nanofibers are in a range between 0.005 to 5 microns in thickness and/or diameter as determined by electron microscopy. In specific embodiments, the sNAG nanofibers are about 0.01, 0.02, 0.03, 0.04, 0.05, 0.06, 0.07, 0.08, 0.09, 0.1, 0.2, 0.25, 0.3, 0.35, 0.4, 0.45, 0.5, 0.55, 0.6, 0.65, 0.7, 0.75, 0.8, 0.85, 0.9, 1, 1.1, 1.2, 1.3, 1.4, 1.5, 1.6, 1.7, 1.8, 1.9, 2, 2.2, 2.4, 2.6, 2.8, 3 or 4 microns in thickness and/or diameter on average, or any range in between (e.g., 0.02 to 2 microns, 0.02 to 1 microns, 0.02 to 0.75 microns, 0.02 to 0.5 microns, 0.02 to 0.5 microns, 0.05 to 1 microns, 0.05 to 0.75 microns, 0.05 to 0.5 microns, 0.1 to 1 microns, 0.1 to 0.75 microns, 0.1 to 0.5 microns, etc.). In specific embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a thickness or diameter of about 0.02 to 1 microns. In other specific embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a thickness or diameter of about 0.05 to 0.5 microns. In specific embodiments, all (100%) of the sNAG nanofibers have a thickness or diameter of about 0.02 to 1 microns or about 0.05 to 0.5 microns. In certain embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a thickness or diameter of about 0.02 to 2 microns, 0.02 to 1 microns, 0.02 to 0.75 microns, 0.02 to 0.5 microns, 0.02 to 0.5 microns, 0.05 to 1 microns, 0.05 to 0.75 microns, 0.05 to 0.5 microns, 0.1 to 1 microns, 0.1 to 0.75 microns, or 0.1 to 0.5 microns.

In certain embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers are between about 1 and about 15 microns in length and have a thickness or diameter of about 0.02 to 1 microns. In certain embodiments, the average length of the sNAG nanofibers is about 1 and about 8 microns or about 1 and about 5 microns in length and the sNAG nanofibers have a thickness or diameter of about 0.02 to 1 microns.

In certain embodiments, the average molecular weight of the sNAG nanofibers is less than 100 kDa, 90 kDa, 80 kDa, 75 kDa, 70 kDa, 65 kDa, 60 kDa, 55 kDa, 50 kDa, 45 kDa, 40 kDa, 35 kDa, 30 kDa, or 25 kDa. In certain embodiments, the average molecular weight of the sNAG nanofibers is between about 10 kDa to 100 kDa, about 20 kDa to 100 kDa, about 10 kDa to 80 kDa, about 20 kDa to 80 kDa, 20 kDa to 75 kDa, about 25 kDa to about 75 kDa, about 30 kDa to about 80 kDa, about 30 kDa to about 75 kDa, about 40 kDa to about 80 kDa, about 40 kDa to about 75 kDa, about 40 kDa to about 70 kDa, about 40 kDa to about 60 kDa, about 40 kDa to about 55 kDa, about 40 kDa to about 50 kDa, about 50 kDa to about 70 kDa, about 50 kDa to about 60 kDa, about 60 kDa to about 80 kDa, about 45 kDa to about 55 kDa, about 50 kDa to about 55 kDa, or about 55 kDa to about 65 kDa. In certain embodiments, the average molecular weight of the sNAG nanofibers is between 50,000 to 100,000 daltons. In certain embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a molecular weight of less than 100 kDa, 90 kDa, 80 kDa, 75 kDa, 70 kDa, 65 kDa, 60 kDa, 55 kDa, 50 kDa, 45 kDa, 40 kDa, 35 kDa, 30 kDa, or 25 kDa. In other embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a molecular weight between about about 10 kDa to 100 kDa, about 20 kDa to 100 kDa, about 10 kDa to 80 kDa, about 20 kDa to 80 kDa, 20 kDa to 75 kDa, about 25 kDa to about 75 kDa, about 30 kDa to about 80 kDa, about 30 kDa to about 75 kDa, about 40 kDa to about 80 kDa, about 40 kDa to about 75 kDa, about 40 kDa to about 70 kDa, about 40 kDa to about 60 kDa, about 40 kDa to about 55 kDa, about 40 kDa to about 50 kDa, about 50 kDa to about 70 kDa, about 50 kDa to about 60 kDa, about 60 kDa to about 80 kDa, about 45 kDa to about 55 kDa, about 50 kDa to about 55 kDa, about 50 kDa to about 70 kDa, about 60 kDa to about 70 kDa or about 55 kDa to about 65 kDa.

In one embodiment, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a molecular weight of 70 kDa±6,000 daltons. In another embodiment, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a molecular weight of about 60 kDa. In another embodiment, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a molecular weight of about 50 kDa. In another embodiment, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a molecular weight of about 40 kDa.

In certain embodiments, 60% to 70%, 60% to 100%, 70% to 100%, 70% to 95%, 70% to 80%, 75% to 80%, 75% to 85%, 85% to 95%, 90% to 95%, 90% to 99% or 95% to 100% of the sNAG nanofibers are acetylated. In some embodiments, 60%, 70%, 75%, 80%, 85%, 90%, 95%, 98%, 99% or 100% of the sNAG nanofibers are acetylated. In other embodiments, more than 70%, 75%, 80%, 85%, 90%, 95%, 98%, 99%, 99.5% or 99.9% of the sNAG nanofibers are acetylated. In some embodiments, equal to or more than 60%, 65%, 70%, 75%, 80%, 85%, 90%, 95% or 99%, or all (100%), of the sNAG nanofibers are acetylated.

In some embodiments, the sNAG nanofibers comprise glucosamine monosaccharides, wherein at least 60%, 65%, 70%, 75%, 80%, 85%, 90%, 95% or 99% of the monosaccharides are N-acetylglucosamine monosaccharides. In other embodiments, the sNAG nanofibers comprise N-acetylglucosamine monosaccharides and glucosamine monosaccharides, wherein at least 60%, 65%, 70%, 75%, 80%, 85%, 90%, 95% or 99% of the monosaccharides are N-acetylglucosamine monosaccharides. In some embodiments, the sNAG nanofibers comprise glucosamine monosaccharide, wherein 60% to 70%, 60% to 100%, 70% to 100%, 70% to 95%, 70% to 90%, 70% to 85%, 70% to 80%, 75% to 80%, 75% to 85%, 85% to 95%, 90% to 95%, 90% to 99% or 95% to 100% of the monosaccharides are N-acetylglucosamine monosaccharides. In other embodiments, the sNAG nanofibers comprise N-acetylglucosamine monosaccharide and glucosamine monosaccharides, wherein 60% to 70%, 60% to 100%, 70% to 100%, 70% to 95%, 70% to 80%, 70% to 90%, 70% to 85%, 75% to 80%, 75% to 85%, 85% to 95%, 90% to 95%, 90% to 99% or 95% to 100% of the monosaccharides are N-acetylglucosamine monosaccharides.

In one aspect, the sNAG nanofibers increase the metabolic rate of serum-starved human umbilical cord vein endothelial cells (“EC”) in a MTT assay. A MTT assay is a laboratory test and a standard colorimetric assay (an assay which measures changes in color) for measuring cellular proliferation (cell growth). Briefly, yellow MTT (3-(4,5-Dimethylthiazol-2-yl)-2,5-diphenyltetrazolium bromide, a tetrazole) is reduced to purple formazan in the mitochondria of living cells. This reduction takes place only when mitochondrial reductase enzymes are active, and therefore conversion can be directly related to the number of viable (living) cells. The metabolic rate of cells may be determined by other techniques commonly known to the skilled artisan.

In another aspect, the sNAG nanofibers do not rescue apoptosis of serum-starved endothelial cells (EC) in a trypan blue exclusion test. A trypan blue exclusion test is a dye exclusion test used to determine the number of viable cells present in a cell suspension. It is based on the principle that live cells possess intact cell membranes that exclude certain dyes, such as trypan blue, Eosin, or propidium, whereas dead cells do not. The viability of cells may be determined by other techniques commonly known to the skilled artisan.

In certain embodiments, compositions comprising the sNAG nanofibers are described, wherein the sNAG nanofibers do one or more of the following: increase the metabolic rate of serum-starved human umbilical cord vein endothelial cells in a MTT assay, or do not rescue apoptosis of serum-starved human umbilical cord vein endothelial cells in a trypan blue exclusion test. In some embodiments, the sNAG nanofibers increase the metabolic rate of serum-starved human umbilical cord vein endothelial cells in a MTT assay and do not rescue apoptosis of serum-starved human umbilical cord vein endothelial cells in a trypan blue exclusion test.

In a specific embodiment, the sNAG nanofibers are biocompatible. Biocompatibility may be determined by a variety of techniques, including, but not limited to such procedures as the elution test, intramuscular implantation, or intracutaneous or systemic injection into animal subjects. Such tests are described in U.S. Pat. No. 6,686,342 (see, e.g., Example 10), which is incorporated by reference herein in its entirety.

In certain embodiments, the sNAG nanofibers used in the methods described herein are non-reactive in a biocompatibility test or tests. For example, the sNAG nanofibers used in the methods described herein may be non-reactive when tested in one, two, or more, or all of the following: an elution test, an intramuscular implantation test, an intracutaneous test, or a systemic test. In other embodiments, the sNAG nanofibers used in the methods described herein have Grade 0 or Grade 1 test score when tested in an elution test, an intramuscular implantation test, an intracutaneous test, or a systemic test. In yet another embodiment, the sNAG nanofibers used in the methods described herein are at most mildly reactive when tested in one, two, or more, or all of the following: an elution test, an intramuscular implantation test, an intracutaneous test, or a systemic test. In certain embodiments, the compositions described herein do not cause an allergenic reaction or an irritation. In other embodiments, the compositions described herein cause at most a mild allergenic reaction or a mild irritation, e.g., at the site of application. The relevant tests and evaluation of test results are described in, e.g., U.S. Pat. Nos. 6,686,342 and 8,858,964, each of which is incorporated herein by reference in its entirety.

In a specific embodiment, the sNAG nanofibers are non-reactive when tested in an intramuscular implantation test. In one aspect, an intramuscular implantation test is an intramuscular implantation test—ISO 4 week implantation, as described in e.g., Section 6.8.3 of U.S. Pat. No. 8,858,964. In certain embodiments, the sNAG nanofibers display no biological reactivity as determined by an elution test (Elution Test Grade=0). In some embodiments, the sNAG nanofibers have a test score equal to “0” and/or are at most a negligible irritant as determined by intracutaneous injection test. In some embodiments, the sNAG nanofibers elicit no intradermal reaction (i.e., Grade I reaction) in Kligman test and/or have a weak allergenic potential as determined by Kligman test.

In certain aspects, the sNAG nanofibers are immunoneutral (i.e., they do not elicit an immune response).

In some embodiments, the sNAG nanofibers are biodegradable. The sNAG nanofibers preferably degrade within about 1 day, 2 days, 3 days, 5 days, 7 days (1 week), 8 days, 10 days, 12 days, 14 days (2 weeks), 17 days, 21 days (3 weeks), 25 days, 28 days (4 weeks), 30 days, 1 month, 35 days, 40 days, 45 days, 50 days, 55 days, 60 days, 2 months, 65 days, 70 days, 75 days, 80 days, 85 days, 90 days, 3 months, 95 days, 100 days or 4 months after administration or implantation into a patient.

In certain embodiments, the sNAG nanofibers do not cause a detectable foreign body reaction. A foreign body reaction, which may occur during wound healing, includes accumulation of exudate at the site of injury, infiltration of inflammatory cells to debride the area, and the formation of granulation tissue. The persistent presence of a foreign body can inhibit full healing. Rather than the resorption and reconstruction that occurs in wound healing, the foreign body reaction is characterized by the formation of foreign body giant cells, encapsulation of the foreign object, and chronic inflammation. Encapsulation refers to the firm, generally avascular collagen shell deposited around a foreign body, effectively isolating it from the host tissues. In one embodiment, treatment of a site (e.g., a wound or a site of a bacterial infection in a wound) with the sNAG nanofibers does not elicit a detectable foreign body reaction in 1 day, 3 days, 5 days, 7 days, 10 days or 14 days after treatment. In one such embodiment, treatment of a site (e.g., a wound) with the sNAG nanofibers does not elicit a foreign body encapsulations in 1 day, 3 days, 5 days, 7 days, 10 days or 14 days after treatment. In one embodiment, application of sNAG nanofibers to the scalp, hair or both of a subject does not elicit a detectable foreign body reaction in 1 day, 3 days, 5 days, 7 days, 10 days or 14 days after treatment. In one such embodiment, application of sNAG nanofibers to the scalp, hair or both of a subject does not elicit a foreign body encapsulations in 1 day, 3 days, 5 days, 7 days, 10 days or 14 days after treatment.

In some embodiments, the sNAG nanofibers: (i) comprise fibers, wherein majority of the fibers are between about 1 and 15 microns in length as determined, e.g., by SEM; and (ii) (a) increase the metabolic rate of serum-starved EC in a MTT assay or do not rescue apoptosis of serum-starved EC in a trypan blue exclusion test or both, and (b) are non-reactive when tested in an intramuscular implantation test. In other embodiments, the sNAG nanofibers: (i) comprise fibers, wherein majority of the fibers are between about 3 and 15 microns in length as determined, e.g., by SEM; and (ii) (a) increase the metabolic rate of serum-starved EC in a MTT assay or do not rescue apoptosis of serum-starved EC in a trypan blue exclusion test or both, and (b) are non-reactive when tested in an intramuscular implantation test. In certain embodiments, the sNAG nanofibers: (i) comprise fibers, wherein majority of the fibers are between about 1 and 12 microns in length as determined, e.g., by SEM; and (ii) (a) increase the metabolic rate of serum-starved EC in a MTT assay or do not rescue apoptosis of serum-starved EC in a trypan blue exclusion test or both, and (b) are non-reactive when tested in an intramuscular implantation test. In certain embodiments, the sNAG nanofibers: (i) comprise fibers, wherein majority of the fibers are between about 4 and 7 microns in length as determined, e.g., by SEM; and (ii) (a) increase the metabolic rate of serum-starved EC in a MTT assay or do not rescue apoptosis of serum-starved EC in a trypan blue exclusion test or both, and (b) are non-reactive when tested in an intramuscular implantation test.

In a specific embodiment, the sNAG nanofibers are obtained by irradiating poly-N-acetylglucosamine or a derivative thereof. See Section 4.1.1, infra, regarding poly-N-acetylglucosamine or derivatives thereof and Section 4.2, infra, regarding methods for producing the sNAG nanofibers using irradiation. Irradiation may be used to reduce the length of poly-N-acetylglucosamine fibers (e.g., poly-β-1→4-N-acetylglucosamine) or poly-N-acetylglucosamine derivative fibers to form shortened poly-N-acetylglucosamine fibers or shortened poly-N-acetylglucosamine derivative fibers, i.e. sNAG nanofibers. Specifically, irradiation may be used to reduce the length and molecular weight of poly-N-acetylglucosamine or a derivative thereof without disturbing its microstructure. The infrared spectrum (IR) of sNAG nanofibers is similar to, about the same as, or equivalent to that of the non-irradiated poly-N-acetylglucosamine or a derivative thereof. In some embodiments, the IR spectrum of the sNAG nanofibers is not statistically different than the IR spectrum of the non-irradiated poly-N-acetylglucosamine or a derivative thereof. In a specific embodiment, the sNAG nanofibers have an IR spectrum that is the same, similar to, or not statistically different than the IR spectrum of fibers of poly-N-acetylglucosamine (e.g., poly-β-1→4-N-acetylglucosamine) with fiber dimensions averaging 20-50 nm×1-2 nm×100 μm and the sNAG nanofibers maintain the microstructure of fibers of poly-N-acetylglucosamine (e.g., poly-β-1→4-N-acetylglucosamine) with fiber dimensions averaging 20-50 nm×1-2 nm×100 μm. In some embodiments, the sNAG nanofibers have a β-1→4 poly-N-acetylglucosamine configuration. In other embodiments, the sNAG nanofibers have a α-1→4 poly-N-acetylglucosamine configuration.

In one embodiment, the sNAG nanofibers are not derived from chitin or chitosan. In another embodiment, the compositions described herein may be derived from chitin or chitosan, or the sNAG nanofibers may be derived from chitin or chitosan. In a specific embodiment, the sNAG nanofibers are derived from microalgae.

4.1.1 Poly-N-Acetylglucosamine and Derivatives Thereof

U.S. Pat. Nos. 5,622,834; 5,623,064; 5,624,679; 5,686,115; 5,858,350; 6,599,720; 6,686,342; 7,115,588 and U.S. Patent Pub. 2009/0117175 (each of which is incorporated herein by reference) describe the poly-N-acetylglucosamine and derivatives thereof, and methods of producing the same. In specific embodiments, the poly-N-acetylglucosamine has a β-1→4 configuration. In other embodiments, the poly-N-acetylglucosamine has a α-1→4 configuration. In some embodiments, the poly-N-acetylglucosamine and derivatives thereof is in the form of a polymer. In specific embodiments, the polymer is in the form of a fiber. In preferred embodiments, the poly-N-acetylglucosamine and derivatives thereof is in the form of a fiber.

Poly-N-acetylglucosamine can, for example, be produced by, and may be purified from, microalgae, preferably diatoms. The diatoms which may be used as starting sources for the production of the poly-N-acetylglucosamine include, but are not limited to members of the Coscinodiscus genus, the Cyclotella genus, and the Thalassiosira genus. Poly-N-acetylglucosamine may be obtained from diatom cultures via a number of different methods, including the mechanical force method and chemical/biological method known in the art (see, e.g., U.S. Pat. Nos. 5,622,834; 5,623,064; 5,624,679; 5,686,115; 5,858,350; 6,599,720; 6,686,342; and 7,115,588, each of which is incorporated herein by reference in its entirety). In certain embodiments, the poly-N-acetylglucosamine is not derived from one or more of the following: a shell fish, a crustacean, an insect, a fungi or yeasts.

In one embodiment, poly-β-1→4-N-acetylglucosamine is derived from a process comprising a) treating a microalgae comprising a cell body and a poly-β-1→4-N-acetylglucosamine polymer fiber with a biological agent (such as hydrofluoric) capable of separating the N-acetylglucosamine polymer fiber from the cell body for a sufficient time so that the poly-β-1→4-N-acetylglucosamine polymer fiber is released from the cell body; b) segregating the poly-β-1→4-N-acetylglucosamine polymer fiber from the cell body; and c) removing contaminants from the segregated poly-β-1→4-N-acetylglucosamine polymer fiber, so that the poly-β-1→4-N-acetylglucosamine polymer is isolated and purified.

In other embodiments, the poly-β-1→4-N-acetylglucosamine may be derived from one or more of the following: a shell fish, a crustacean, an insect, a fungi or yeasts. In certain embodiments, the compositions described herein do not comprise chitin or chitosan. In some embodiments, the poly-β-1→4-N-acetylglucosamine is not derived from one or more of the following: a shell fish, a crustacean, an insect, a fungi or yeasts.

In certain embodiments, a poly-N-acetylglucosamine composition comprises 60% to 70%, 60% to 100%, 70% to 100%, 70% to 95%, 70% to 80%, 75% to 80%, 75% to 85%, 85% to 95%, 90% to 95%, 90% to 99% or 95% to 100% of acetylated glucosamine (i.e., N-acetylglucosamine) monosaccharides. In some embodiments, a poly-N-acetylglucosamine composition comprises 60%, 70%, 75%, 80%, 85%, 90%, 95%, 98%, 99% or 100% of acetylated glucosamine (i.e., N-acetylglucosamine) monosaccharides. In other embodiments, a poly-N-acetylglucosamine composition comprises more than 60%, 70%, 75%, 80%, 85%, 90%, 95%, 98%, 99%, 99.5% or 99.9% of the acetylated glucosamine (i.e., N-acetylglucosamine). In some embodiments, a poly-N-acetylglucosamine composition comprises equal to or more than 60%, 65%, 70%, 75%, 80%, 85%, 90%, 95% or 99%, or all (100%), of the acetylated glucosamine (i.e., N-acetylglucosamine).

In some embodiments, a poly-N-acetylglucosamine composition comprises glucosamine monosaccharides, wherein at least 60%, 65%, 70%, 75%, 80%, 85%, 90%, 95% or 99% of the monosaccharides are N-acetylglucosamine monosaccharides. In other embodiments, a poly-N-acetylglucosamine composition comprises N-acetylglucosamine monosaccharides and glucosamine monosaccharides, wherein at least 60%, 65%, 70%, 75%, 80%, 85%, 90%, 95% or 99% of the monosaccharides are N-acetylglucosamine monosaccharides. In some embodiments, a poly-N-acetylglucosamine composition comprises glucosamine monosaccharides, wherein 60% to 70%, 60% to 100%, 70% to 100%, 70% to 95%, 70% to 80%, 75% to 80%, 75% to 85%, 85% to 95%, 90% to 95%, 90% to 99% or 95% to 100% of the monosaccharides are N-acetylglucosamine monosaccharides. In other embodiments, a poly-N-acetylglucosamine composition comprises N-acetylglucosamine monosaccharides and glucosamine monosaccharides, wherein 60% to 70%, 60% to 100%, 70% to 100%, 70% to 95%, 70% to 80%, 75% to 80%, 75% to 85%, 85% to 95%, 90% to 95%, 90% to 99% or 95% to 100% of the monosaccharides are N-acetylglucosamine monosaccharides.

Derivatives of poly-N-acetylglucosamine may also be used in a composition described herein. Derivatives of poly-N-acetylglucosamine and methods of making such derivatives are described in U.S. Pat. No. 5,623,064 (see, e.g., Section 5.4), which is incorporated by reference herein in its entirety. Poly-N-acetylglucosamine may be derivatized by being sulfated, phosphorylated and/or nitrated. Poly-N-acetylglucosamine derivatives include, e.g., sulfated poly-N-acetylglucosamine derivatives, phosphorylated poly-N-acetylglucosamine derivatives, or nitrated poly-N-acetylglucosamine derivatives. Additionally, one or more of the monosaccharide units of the poly-N-acetylglucosamine may contain one or more sulfonyl groups one or more O-acyl groups. One or more of the monosaccharides of the poly-N-acetylglucosamine, may contain an O-alkyl group. One or more of the monosaccharide units of the poly-N-acetylglucosamine may be an alkali derivative. In one embodiment, the poly-N-acetylglucosamine is derivatized with lactic acid. Wherein, in another embodiment, the derivative is not derivatized with lactic acid. In a specific embodiment, derivatives of poly-N-acetylglucosamine are not used to produce sNAG nanofibers. In some embodiments, a derivative of poly-N-acetylglucosamine is low molecular weight polymer, which is soluble in low pH. In certain embodiments, a derivative of poly-N-acetylglucosamine is a small particle (1 to 10 μm)/low molecular weight polymer that is insoluble in low pH.

4.2 Methods of Making sNAG Nanofibers

The poly-N-acetylglucosamine fibers, and any derivatives of poly-N-acetylglucosamine fibers described above, can be irradiated as dry fibers or fiber membranes. Alternatively, poly-N-acetylglucosamine fibers, and any derivatives of poly-N-acetylglucosamine fibers described above, can be irradiated when wet. The methods of making sNAG nanofibers by irradiation and the sNAG nanofibers so produced have been described in U.S. Pat. Nos. 8,871,247, 9,139,663, and 9,139,664, each of which is incorporated by reference herein in its entirety.

In certain embodiments, the poly-N-acetylglucosamine fibers are formulated into a suspension/slurry or wet cake for irradiation. Irradiation can be performed prior to, concurrently with or following the formulation of the fibers into its final formulation, such as a dressing. Generally, the fiber content of suspensions/slurries and wet cakes can vary, for example from about 0.5 mg to about 50 mg of fiber per 1 ml of distilled water are used for slurries and from about 50 mg to about 1000 mg of fiber per 1 ml of distilled water are used for wet cake formulations. The fiber may first be lyophilized, frozen in liquid nitrogen, and pulverized, to make it more susceptible to forming a suspension/slurry or wet cake. Also, the suspensions/slurries can be filtered to remove water such that a wet cake is formed. In certain aspects, the fiber is irradiated as a suspension comprising about 0.5 mg, 1 mg, 2 mg, 3 mg, 4 mg, 5 mg, 6 mg, 7 mg, 8 mg, 9 mg, 10 mg, 12 mg, 15 mg, 18 mg, 20 mg, 25 mg or 50 mg of polymer or fiber per ml of distilled water, or any range in between the foregoing embodiments (e.g., 1-10 mg/ml, 5-15 mg/ml, 2-8 mg/ml, 20-50 mg/ml, etc.). In other aspects, the fiber is irradiated as a wet cake, comprising about 50-1,000 mg fiber per 1 ml of distilled water. In specific embodiments, the wet cake comprises about 50, 100, 200, 300, 400, 500, 600, 700, 800, 900 or 1000 mg of fiber per 1 ml distilled water, or any range in between (e.g., 100-500 mg/ml, 300-600 mg/ml, 50-1000 mg/ml, etc.).

The irradiation is preferably in the form of gamma radiation, e-beam radiation, or x-rays. Two sources of irradiation are preferred: radioactive nuclides and electricity. In specific embodiment, the radioactive nuclides are cobalt-60 and cesium-137. Both of these nuclides emit gamma rays, which are photons containing no mass. The gamma rays have energies from 0.66 to 1.3 MeV. Using electricity, electrons are generated and accelerated to energies up to 10 MeV or higher. When irradiating fibers to reduce their size, a consideration to take into account is that the depth of penetration of materials with densities similar to water by 10 MeV electrons is limited to about 3.7 cm with one-sided exposure or about 8.6 cm with two-sided exposure. Depth of penetration decreases at lower electron energies. Electron energy can be converted to x-rays by placing a metal (usually tungsten or tantalum) target in the electron beam path. Conversion to x-rays is limited to electrons with energies up to 5 MeV. X-rays are photons with no mass and can penetrate fibers similar to gamma rays. There is only about 8% efficiency in the conversion of electron energy to x-ray energy. High powered electron beam machines are needed in x-ray production facilities to account for the low conversion efficiency.

In a specific embodiment, the irradiation is gamma irradiation.

The absorbed dose of radiation is the energy absorbed per unit weight of product, measured in gray (gy) or kilogray (kgy). For dried fibers, the preferred absorbed dose is about 500-2,000 kgy of radiation, most preferably about 750-1,250 kgy or about 900-1,100 kgy of radiation. For wet fibers, the preferred absorbed dose is about 100-500 kgy of radiation, most preferably about 150-250 kgy or about 200-300 kgy of radiation. In a specific embodiment, wet fibers are irradiated at 300 kgy.

The dose of radiation can be described in terms of its effect on the length of the fibers. In specific embodiments, the dose of radiation used preferably reduces the length of the fiber by anywhere from about 10% to 90% of the starting length of the fiber, respectively. In specific embodiments, the average length is reduced by about 10%, by about 20%, by about 30%, by about 40%, by about 50%, by about 60%, by about 70%, by about 80%, or by about 90%, or any range in between (e.g., 20-40%, 30-70%, and so on and so forth). Alternatively, the dose of radiation used preferably reduces the length of the fiber to anywhere from 1 to 30 microns. In specific embodiments, and depending on the starting fiber length, the average length of the fiber is reduced to less than about 20 microns, less than about 15 microns, less than about 14 microns, less than about 13 microns, less than about 12 microns, less than about 11 microns, less than about 10 microns, less than about 8 microns, less than about 7 microns, less than about 5 microns, less than about 4 microns, less than about 3 microns, less than 2 microns, or less than 1 microns. In certain embodiments, the length of the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the fibers is reduced to no greater than about 20 microns, no greater than about 15 microns, no greater than about 12 microns, no greater than about 10 microns, no greater than about 8 microns, no greater than about 7 microns, or no greater than about 5 microns. In certain embodiments, irradiation of the fibers reduces the length of the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the fibers to anywhere between about 1 to 20 microns, between about 1 to 15 microns, between about 2 to 15 microns, between about 3 to 15 microns, between about 1 to 12 microns, between about 2 to 12 microns, between about 1 to 10 microns, between about 2 to 10 microns, between about 1 to 8 microns, between about 2 to 8 microns, between about 1 to 7 microns, between about 2 to 7 microns, between about 3 to 8 microns, between about 4 to 7 microns, between about 1 to 5 microns, between about 2 to 5 microns, between about 3 to 5 microns, between about 4 to 10 microns, or any ranges between the foregoing lengths, which are also encompassed.

In certain embodiments, the average length of the fibers is reduced to no greater than about 20 microns, no greater than about 15 microns, no greater than about 12 microns, no greater than about 10 microns, no greater than about 8 microns, no greater than about 7 microns, or no greater than about 5 microns. In other embodiments, the average length of the fibers is reduced to no less than 1 micron. In certain embodiments, irradiation of the fibers reduces the average length of the fibers to anywhere between about 1 to 20 microns, between about 1 to 15 microns, between about 2 to 15 microns, between about 3 to 15 microns, between about 1 to 12 microns, between about 2 to 12 microns, between about 1 to 10 microns, between about 2 to 10 microns, between about 1 to 8 microns, between about 2 to 8 microns, between about 1 to 7 microns, between about 2 to 7 microns, between about 3 to 8 microns, between about 4 to 7 microns, between about 1 to 5 microns, between about 2 to 5 microns, between about 3 to 5 microns, between about 4 to 10 microns, or any ranges between the foregoing lengths, which are also encompassed. In a specific embodiment, the length of the fibers is determined by SEM.

In certain embodiments, the length of the fibers is reduced to no greater than about 20 microns, no greater than about 15 microns, no greater than about 12 microns, no greater than about 10 microns, no greater than about 8 microns, no greater than about 7 microns, or no greater than about 5 microns, as determined by any method known to one skilled in the art, for example, by SEM. In other embodiments, the length of the fibers is reduced to no less than 1 micron, as determined by any method known to one skilled in the art, for example, by SEM. In certain embodiments, irradiation of the fibers reduces the length of the fibers to anywhere between about 1 to 20 microns, between about 1 to 15 microns, between about 2 to 15 microns, between about 3 to 15 microns, between about 1 to 12 microns, between about 2 to 12 microns, between about 1 to 10 microns, between about 2 to 10 microns, between about 1 to 8 microns, between about 2 to 8 microns, between about 1 to 7 microns, between about 2 to 7 microns, between about 3 to 8 microns, between about 4 to 7 microns, between about 1 to 5 microns, between about 2 to 5 microns, between about 3 to 5 microns, between about 4 to 10 microns, or any ranges between the foregoing lengths, which are also encompassed, as determined by any method known to one skilled in the art, for example, by SEM.

The dose of radiation can also be described in terms of its effect on the molecular weight of the fiber. In specific embodiments, the dose of radiation used preferably reduces the molecular weight of the fiber by anywhere from about 10% to 90% of the starting weight of the fiber. In specific embodiments, the average molecular weight is reduced by about 10%, by about 20%, by about 30%, by about 40%, by about 50%, by about 60%, by about 70%, by about 80%, or by about 90%, or any range in between (e.g., 20-40%, 30-70%, and so on and so forth). Alternatively, the dose of radiation used preferably reduces the molecular weight of the fiber to anywhere from 1,000 to 1,000,000 daltons. In specific embodiments, and depending on the starting molecular weight, the average molecular weight of the fiber is reduced to less than 1,000,000 daltons, less than 750,000 daltons, less than 500,000 daltons, less than 300,000 daltons, less than 200,000 daltons, less than 100,000 daltons, less than 90,000 daltons, less than 80,000 daltons, less than 70,000 daltons, less than 60,000 daltons, less than 50,000 daltons, less than 40,000 daltons, less than 25,000 daltons, less than 10,000 daltons, or less than 5,000 daltons. In certain embodiments, the average molecular weight is reduced to no less than 500 daltons, no less than 1,000 daltons, no less than 2,000 daltons, no less 3,500 daltons, no less than 5,000 daltons, no less than 7,500 daltons, no less than 10,000 daltons, no less than 25,000 daltons, no less than 40,000 daltons, no less than 50,000 daltons, no less than 60,000 daltons or no less than 100,000 daltons. Any ranges between the foregoing average molecular weights are also encompassed; for example, in certain embodiments, irradiation of the fiber reduces the average molecular weight to anywhere between 10,000 to 100,000 daltons, between 1,000 and 25,000 daltons, between 50,000 to 100,000 daltons, between 50,000 and 500,000 daltons, between 25,000 and 100,000 daltons, between 30,000 and 90,000 daltons, between about 40,000 and 80,000 daltons, between about 40,000 and 60,000 daltons, between about 25,000 and 75,000 daltons, between about 50,000 and 70,000 daltons, between about 60,000 and 80,000 daltons, or between about 55,000 and 65,000 daltons and so on and so forth. In certain embodiments, irradiation of the fibers reduces the molecular weight of the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the fibers to anywhere between about 20,000 and 100,000 daltons, about 25,000 and 75,000 daltons, about 30,000 and 90,000 daltons, about 40,000 and 80,000 daltons, about 50,000 and 70,000 daltons, about 60,000 daltons to about 80,000 daltons, about 40,000 and 60,000 daltons, or about 55,000 and 65,000 daltons. In certain embodiments, irradiation of the fibers reduces the molecular weight of the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the fibers to about 70,000 daltons±6,000 daltons. In certain embodiments, irradiation of the fibers reduces the molecular weight of the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the fibers to about 70,000 daltons±6,000 daltons. In certain embodiments, irradiation of the fibers reduces the molecular weight of the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the fibers to about 70,000 daltons.

In a specific embodiment, irradiation of fibers reduces the average fiber length to 1 to 8 μm or 1 to 5 μm and average molecular weight to 50,000 to 100,000 daltons (e.g., 70,000±6,000 daltons).

Following irradiation, slurries can be filtered and dried, and wet cakes can be dried, to form compositions that are useful in methods described herein.

4.3 Compositions Comprising sNAG Nanofibers

A composition comprising sNAG nanofibers described herein may be formulated as a cream, a membrane, a film, a liquid solution, a suspension, a powder, a paste, an ointment, a gelatinous composition, an aerosol, a serum, a gel, or a spray. In one embodiment, a composition comprising sNAG nanofibers described herein is formulated as an ultra-thin membrane. In a specific embodiment, a composition comprising sNAG nanofibers described herein is formulated as a serum, suspension or a gel. In a specific embodiment, the composition is a serum. The composition described herein may be used in the methods described herein.

In certain embodiments, a composition comprising sNAG nanofibers described herein is not formulated as a shampoo, conditioner or lotion. In other embodiments, a composition comprising sNAG nanofibers described herein is formulated as a shampoo, conditioner or lotion.

In specific embodiments, a composition comprising sNAG nanofibers described herein does not include any one, two, three, or more, or all of the following: a liposome, diethyleneglycole monoethyletere, microcapsules, a nanoparticle or microcapsules coated with sNAG fibers, a nanoparticle or microcapsule encapsulated with sNAG nanofibers, water-in-oil emulsion, oil-in-water emulsion, and a water-in-silicone oil emulsion.

A composition comprising sNAG nanofibers described herein may include one or more acceptable excipients. Suitable excipients may include water, saline, salt solution, dextrose, glycerol, ethanol and the like, or combinations thereof. Suitable excipients also include starch, glucose, lactose, sucrose, gelatin, malt, rice, flour, chalk, silica gel, sodium stearate, glycerol monostearate, oil (including those of petroleum, animal, vegetable or synthetic origin, such as peanut oil, soybean oil, mineral oil, sesame oil and the like), talc, sodium chloride, dried skim milk, propylene, glycol and the like. In addition, a composition comprising sNAG nanofibers may include one or more of wetting agents, emulsifying agents, pH buffering agents, and other agents. The sNAG nanofiber compositions may also be incorporated in a physiologically acceptable carrier.

In a specific embodiment, a composition comprising sNAG nanofibers includes purified water. In another specific embodiment, a composition comprising sNAG nanofibers includes purified water and one or more preservatives (e.g., phenoxyethanol, caprylyl glycol, phenethyl alcohol, pentylene glycol or propanediol). In another specific embodiment, a composition comprising sNAG nanofibers includes purified water, phenoxyethanol, caprylyl glycol, and sodium hydroxide. In another specific embodiment, a composition comprising sNAG nanofibers includes purified water, phenoxyethanol 0.6%, caprylyl glycol, and sodium hydroxide. In specific embodiments, a composition comprising sNAG nanofibers does not include any one or all of the following: sulfates, parabens, silicones, and sodium chlorides. In another specific embodiment, a composition comprising sNAG nanofibers includes purified water, phenoxyethanol, caprylyl glycol, and sodium hydroxide, and the composition does not include sulfates, parabens, silicones, and sodium chlorides. In a specific embodiment, a composition comprising sNAG nanofibers is one described in Section 5, infra.

In certain embodiments, a composition comprising sNAG nanofibers described herein does not contain one, two, three or more, or any of the following agents: a sulfa agent (e.g., sulfamonomethoxine, acetyl sulfamethoxazole, salazosulfapyridine, sulfadiazine, silver sulfadiazine, sulfadimethoxine, sulfathiazole, sulfaphenazole, sulfamethoxazole, sulfamethoxypyridazine, sulfamethopyrazine, sulfametomidine, sulfamethizole, sulfamerazine, sulfisoxazole, sulfisomidine, sulfisomidine sodium, homosulfamine and a derivative thereof); a fluorinated acid; an aldehyde-bearing compound or organic acid; a silicone surfactant (e.g., siloxane or polyoxyalkylene copolymers); silicone; epoxy-functionalized inorganic oxide particles; inorganic particulates; organic polymeric particulates; pigments; lakes; silicon-based particulates; silicon polymer (e.g., polydimethylsiloxane; dimethyl siloxane-glycol copolymer; cyclic dimethylpolysiloxane; α-hydroxy-omega-hydroxy-polyoxydimethylsilylene; dimethyl siloxane-aminoalkylsiloxane copolymer with hydroxy end groups; monomethylpolysiloxane having lauryl side chains and polyoxyethylene end chains; monomethylpolysiloxane having lauryl side chains and polyoxypropylene end chains; monomethylpolysiloxane having lauryl side chains, polyoxyethylene end chains and polyoxypropylene end chains; dimethyl siloxane-aminoalkylsiloxane copolymer with trimethylsilyl end groups; dimethylsiloxane-glycol copolymer acetate and trimethyl(octadecyloxy)silane); elastomeric silane or siloxane; polyvinylpyrrolidone/vinyl acetate copolymer; terpolymer made from vinyl pyrrolidone, vinyl caprolactam and a basic acrylamide monomer; amino acids obtained from the hydrolysis of a keratin; keratin; hydrolyzed keratin; a 2,5-diamino-6-nitropyridine derivative; minoxidil; dimethyl sulfoxide; ampholytic copolymerizate; soluble β-(1,3) glucans (including, e.g., water soluble β-(1,3) glucans, substantially free from β-(1,6) linkages); polyvinyl pyrrolidone; vinyl pyrrolidone-vinyl acetate copolymers; vinyl pyrrolidone-vinyl acetate-vinyl propionate terpolymers; polyacrylamides; polyvinyl alcohols; polyethyene glycols; anti-microbial oligoaminosaccharide (e.g., anti-microbial oligoaminosaccharide having anti-dandruff activity); lignin (e.g., sulfur-free lignin, hydroxypropyl lignin, hydroxybutyl lignin, dihydroxypropyl lignin or mixtures thereof); hair dye; thickening agent; additional polymers (e.g., anionic polymers, cationic polymers, amphoteric polymers, zwitterionic polymers and nonionic polymers); a 2,6-dinitro-phenol derivative; a hair conditioning agent (e.g., silicones, fatty alcohols, oils, panthenol, panthenyl ethyl ether, sorbitol, betaine, creatine or protein hydrolysates); N-(carboxymethylidene)allantoin; a polyphenol derivative (e.g., phloroglucinol derivatives and tannin derivatives); cyclodextrin; arabinogalactan; branched sulphonic polyester; (meth)acrylic thickening polymer; an anti-bacterial agent, an anti-fungal; an anti-viral agent; steroids; finasteride; spinolactone; flutamide; ketoconazole; and pyrithione zinc. In certain embodiments, a sNAG nanofiber composition described herein does not comprise cells (e.g., fibroblasts or epithelial cells), human fibroblast derived dermal substitute, or growth factor to promote tissue regeneration. In certain embodiments, a sNAG nanofiber composition described herein does not comprise any of the following: cells (e.g., fibroblasts or epithelial cells), human fibroblast derived dermal substitute, growth factor to promote tissue regeneration, and any of the agents in the first sentence of this paragraph.

In certain embodiments, a sNAG nanofiber composition described herein does not comprise any of the following: platelet derived growth factor (PDGF), fibroblast growth factor (FGF) (e.g., acidic or basic FGF, or both), bone morphogenetic protein(s) (BMPs) (e.g., BMP-2), insulin-like growth factor I, insulin-like growth factor II, transforming growth factor β (TGF-β), keratinocyte growth factor, RNAi, or gene therapy.

In certain embodiments, a composition comprising the sNAG nanofibers does not contain either chitin (e.g., chitin glycan) or chitosan (e.g., N-(carboxymethylidene) chitosan, N-hydroxyalkyl-O-benzyl chitosan or glycated chitosan). In some embodiments, a composition comprising the sNAG nanofibers does not contain one, two, three or more, or any of the agents in the two preceding paragraphs (i.e., paragraphs [0081] and [0082]) and does not contain chitin (e.g., chitin glycan), chitosan (e.g., N-(carboxymethylidene) chitosan, N-hydroxyalkyl-O-benzyl chitosan or glycated chitosan), cells and human derived dermal substitute.

In certain embodiments, a composition comprising the sNAG nanofibers does not contain glucosamine. In some embodiments, a composition comprising the sNAG nanofibers does not contain either chitin (e.g., chitin glycan), chitosan (e.g., N-(carboxymethylidene) chitosan, N-hydroxyalkyl-O-benzyl chitosan or glycated chitosan), or glucosamine. In certain embodiments, a composition comprising the sNAG nanofibers does not contain one, two, three or more, or any of the agents in the paragraphs [0081] and [0082]) and does not contain chitin (e.g., chitin glycan), chitosan (e.g., N-(carboxymethylidene) chitosan, N-hydroxyalkyl-O-benzyl chitosan or glycated chitosan), glucosamine, cells and human derived dermal substitute.

In another specific embodiment, a composition comprising the sNAG nanofibers includes purified water, phenoxyethanol, caprylyl glycol, and sodium hydroxide and does not include sulfates, parabens, silicones, sodium chlorides, chitin (e.g., chitin glycan), chitosan (e.g., N-(carboxymethylidene) chitosan N-hydroxyalkyl-O-benzyl chitosan or glycated chitosan), glucosamine, cells, human derived dermal substitute, and any of the agents referenced in paragraphs [0081] and [0082] above. In another specific embodiment, a composition consists or consists essentially of sNAG nanofibers, purified water, phenoxyethanol, caprylyl glycol, and sodium hydroxide. In another specific embodiment, a composition consists or consists essentially of sNAG nanofibers, purified water, phenoxyethanol 0.6%, caprylyl glycol, and sodium hydroxide.

In another specific embodiment, a composition comprising the sNAG nanofibers includes purified water, glucosamine, phenoxyethanol, caprylyl glycol, and sodium hydroxide. In specific embodiments, a composition comprising the sNAG nanofibers does not include any one or all of the following: sulfates, parabens, silicones, and sodium chlorides. In another specific embodiment, a composition comprising the sNAG nanofibers includes purified water, glucosamine, phenoxyethanol, caprylyl glycol, and sodium hydroxide, and the composition does not include sulfates, parabens, silicones, and sodium chlorides. In another specific embodiment, a composition comprising the sNAG nanofibers includes purified water, glucosamine, phenoxyethanol, caprylyl glycol, and sodium hydroxide and does not include sulfates, parabens, silicones, sodium chlorides, chitin (e.g., chitin glycan), chitosan (e.g., N-(carboxymethylidene) chitosan, N-hydroxyalkyl-O-benzyl chitosan or glycated chitosan), cells, human derived dermal substitute, and any of the agents referenced in paragraphs [0081] and [0082] above. In another specific embodiment, a composition consists or consists essentially of sNAG nanofibers, purified water, glucosamine, phenoxyethanol, caprylyl glycol, and sodium hydroxide. In certain embodiments, the glucosamine used in the sNAG nanofiber composition is D(+)-glucosamine, glucosamine hydrochloride, N-acetyl glucosamine, or glucosamine sulfate. In a specific embodiment, glucosamine is present in the composition at a concentration of 1 mg/mL to 10 mg/mL. In a specific embodiment, the glucosamine is D-(+)-glucosamine hydrochloride. In another embodiment, the glucosamine is non-animal derived (e.g., plant derived).

In certain aspects, the sNAG nanofiber is the only active ingredient in a composition. In certain embodiments, a sNAG nanofiber composition does not comprise any additional therapy or agent. In some embodiments, a sNAG nanofiber composition does not comprise any additional therapy or agent for the hair or scalp.

In some embodiments, a sNAG nanofiber composition comprises one or more additional active ingredients, e.g., to promote an anti-bacterial effect, an anti-viral effect, anti-fungal effect or a combination thereof. In some embodiments, the one or more additional active ingredients promotes an anti-bacterial effect. In some embodiments, the one or more additional active ingredients promotes an anti-viral effect. In other embodiments, the one or more additional active ingredient promotes an anti-fungal effect.

In certain embodiments, a sNAG nanofiber composition comprises one or more additional active ingredients to promote any or all of the following: hair growth, hair thickness, hair body, hair health. In some embodiments, a sNAG nanofiber composition comprises one or more of the agents referenced in paragraphs [0081] and/or [0082]. In certain embodiments, a sNAG nanofiber comprises one or more of the agents referenced in paragragh [0081]. In some embodiments, a sNAG nanofiber composition comprises one, two or more, or all of the following additional active ingredients: hair penetration enhancers, hair growth promoters, circulation promoter, and anti-inflammatories. In certain embodiments, a sNAG nanofiber composition does not comprise one, two or more, or all of the following additional active ingredients: hair penetration enhancers, hair growth promoters, circulation promoter, and anti-inflammatories.

In some embodiments, a sNAG nanofiber composition comprises one, two, or more, or all of the following ingredients: Methylsulfonylmethane, Copper Tripeptide-1, Curcuma Longa Peptides, Caffeine, Biotin, Album (Sandalwood) Wood Extract, Pisum Sativum (Pea) Sprout Extract, and Keratinocyte Growth Factor. In certain embodiments, a sNAG nanofiber composition does not comprise one, two, or more, or all of the following ingredients: Methylsulfonylmethane, Copper Tripeptide-1, Curcuma longa Peptides, Caffeine, Biotin, Album (Sandalwood) Wood Extract, Pisum sativum (Pea) Sprout Extract, and Keratinocyte Growth Factor.

In some embodiments, a sNAG nanofiber composition described herein does not comprise one or more additional active ingredients, e.g., to promote an anti-bacterial effect, an anti-viral effect, anti-fungal effect or a combination thereof. In some embodiments, a sNAG nanofiber composition described herein does not comprise one or more additional active ingredients that promotes an anti-bacterial effect. In some embodiments, a sNAG nanofiber composition described herein does not comprise one or more additional active ingredients that promotes an anti-viral effect. In other embodiments, a sNAG nanofiber composition described herein does not comprise one or more additional active ingredients that promotes an anti-fungal effect.

In certain embodiments, a sNAG nanofiber composition described herein does not comprise one, two or more, or all of the following additional active ingredients: hair growth promoter, hair thickness promoter, hair body promoter, hair health promoter, hair penetration promoter, circulation promoter, and anti-inflammatory agent.

In other aspects, a sNAG nanofiber composition does not comprise a significant amount of protein material. In specific embodiments, the protein content of a sNAG nanofiber composition is no greater than 0.1%, 0.5% or 1% by weight. In other embodiments, the protein content of the composition is undetectable by Coomassie staining.

The final amount of the sNAG nanofibers in a composition may vary. For example, the amount of the sNAG nanofibers in a composition (e.g., prepared for administration to a patient) may be greater than or equal to about 50%, about 60%, about 70%, about 75%, about 80%, about 85%, about 90%, about 95%, about 98%, or about 99% weight by volume. In one embodiment, the amount of the sNAG nanofibers in a composition is about 95%, about 98%, about 99, or about 100%. Also, the amount of the sNAG nanofibers in a composition (e.g., prepared for administration to a patient) may be about 50%-100%, about 60%-100%, about 70%-100%, about 75%-100%, about 80%-100%, about 90%-100%, about 95%-100%, about 70%-95%, about 75%-95%, about 80%-95%, about 90%-95%, about 70%-90%, about 75%-90%, or about 80%-90% weight/volume. A composition may comprise more than 30%, 40%, 50%, 60%, 70%, 75%, 80%, 90%, 95% or 99% solution of the sNAG nanofibers.

In some embodiments, a composition described herein (e.g., a serum, a suspension or a gel) comprises approximately 0.05 mg to approximately 50 mg, approximately 0.05 mg to approximately 40 mg, approximately 0.05 mg to approximately 30 mg, approximately 0.05 mg to approximately 20 mg, or approximately 0.05 mg to approximately 10 mg of sNAG nanofibers per mL of isotonic solution (e.g., saline or PBS) or water. In some embodiments, a composition described herein (e.g., a serum, a suspension or a gel) comprises approximately 0.05 mg to approximately 5 mg of sNAG nanofibers per mL of isotonic solution (e.g., saline or PBS) or water. In some embodiments, a composition described herein (e.g., a serum, a suspension or a gel) comprises approximately 0.05 mg to approximately 3 mg of sNAG nanofibers per mL of isotonic solution (e.g., saline or PBS) or water. In some embodiments, a composition described herein (e.g., a serum, a suspension or a gel) comprises approximately 0.5 to approximately 5 mg of sNAG nanofibers per mL of isotonic solution (e.g., saline or PBS) or water. In some embodiments, a composition described herein (e.g., a serum, a suspension or a gel) comprises approximately 0.5 mg to approximately 3 mg of sNAG nanofibers per mL of isotonic solution (e.g., saline or PBS) or water. In some embodiments, a composition described herein (e.g., a serum, a suspension or a gel) comprises approximately 0.5 mg to 2 mg of sNAG nanofibers per mL of isotonic solution (e.g., saline or PBS) or water. In some embodiments, a composition described herein (e.g., a serum, a suspension or a gel) comprises approximately 1 mg to 2 mg of sNAG nanofibers per mL of isotonic solution (e.g., saline or PBS) or water.

In some embodiments, the concentration of sNAG nanofibers in a composition described herein (e.g., a serum, suspension or gel) is about 0.05-5 mg/mL. In some embodiments, the concentration of sNAG nanofibers in a composition described herein (e.g., a serum, suspension or gel) is about 0.05-3 mg/mL. In some embodiments, the concentration of sNAG nanofibers in a composition described herein (e.g., a serum, suspension or gel) is about 0.5-3 mg/mL. In other embodiments, the concentration of sNAG nanofibers in a composition described herein (e.g., a serum, suspension or gel) is about 1-5 mg/mL. In other embodiments, the concentration of sNAG nanofibers in a composition described herein (e.g., a serum, suspension or gel) is about 1-2 mg/mL. In other embodiments, the concentration of sNAG nanofibers in a composition described herein (e.g., a serum, suspension or gel) is about 1 mg/mL or 1.5 mg/mL.

In a specific embodiment, a composition described herein has the pH of the natural pH of hair within ±1 or 1.5. In another specific embodiment, a composition described herein has pH 6 to 7, 4.5 to 7, 4.5 to 6, or 4.5 to 5.5. In another specific embodiment, a composition described herein has a pH of 4.5, 5, 5.5, 6, 6.5, or 7.

In a specific embodiment, a composition described herein meets the strength specifications and maximum impurity limit indicated by the Food Chemicals Codex.

In other embodiments, a composition described herein contains a preservative to prevent microbial growth and degradation. In other embodiments, a composition described herein contains a preservative to prevent bacterial growth. In some embodiments, a composition described herein contains no endotoxins.

4.4 Use of Compositions

The sNAG nanofibers described herein or a composition thereof may be used in accordance with the methods described herein or in the kits described herein. See, e.g., Sections 4.3 and 5 for compositions that may be used in the methods described herein. In one aspect, provided herein are methods for reducing hair shedding of a subject (e.g., a human) using sNAG nanofibers or a composition thereof. In one embodiment, provided herein is a method for reducing hair shedding, the method comprising administering to the scalp, hair or both of a subject (e.g., a human) a composition comprising sNAG nanofibers.

In another aspect, provided herein are methods for improving hair growth of a subject (e.g., a human) using sNAG nanofibers or a composition thereof. In one embodiment, provided herein is a method for improving hair growth, the method comprising administering to the scalp, hair or both of a subject (e.g., a human) a composition comprising sNAG nanofibers. In certain embodiments, the hair grows at a 10%, 15%, 20%, 25%, 30%, 35%, 40%, 50%, 55% or greater rate after treatment with the composition than the rate of hair growth observed prior to treatment with the composition. In some embodiments, the hair grows at a 35% to 40%, 35% to 50%, 40% to 55% greater rate after treatment with the composition than the rate of hair growth observed prior to treatment with the composition. In certain embodiments, the hair grows at a 10%, 15%, 20%, 25%, 30%, 35%, 40%, 50%, 55% or greater rate after treatment with the composition than the rate of hair growth observed in untreated subjects who are comparable in health (e.g., scalp and hair health) to the treated subject. In some embodiments, the hair grows at a 35% to 40%, 35% to 50%, or 40% to 55% greater rate after treatment with the composition than the rate of hair growth observed in untreated subjects who are comparable in health (e.g., scalp and hair health) to the treated subject. In certain embodiments, the average rate of hair growth after treatment is about 15.10 cm, about 15.20 cm, about 15.3 cm, about 15.4 cm, about 15.5 cm, about 15.6 cm, about 15.7 cm, about 15.8 cm, about 15.9 cm, about 16 cm, about 16.1 cm, about 16.2 cm, or about 16.3 cm per year. In some embodiments, the average rate of hair growth after treatment is about 1.30 cm, about 1.35 cm, about 1.4 cm, about 1.45 cm, about 1.5 cm, about 1.55 cm, about 1.6 cm, about 1.65 cm, about 1.7 cm, about 1.75 cm, about 1.8 cm, about 1.85 cm, or about 2 cm per month. Typically, the rate or speed of hair growth is about 1.25 cm per month, or about 15 cm per year.

In another aspect, provided herein are methods for thickening existing hair fiber diameter of a subject (e.g., a human) using sNAG nanofibers or a composition thereof. In one embodiment, provided herein is a method for thickening existing hair fiber diameter of a subject (e.g., a human), the method comprising administering to the scalp, hair or both of the subject a composition comprising sNAG nanofibers. In certain embodiments, the hair fiber thickens in diameter by 10%, 15%, 20%, 25%, 30%, 35%, 40%, 50%, 55% or more after treatment with the composition than the hair fiber thickens in diameter prior to treatment with the composition. In some embodiments, the hair fiber thickens in diameter by 35% to 40%, 35% to 50%, or 40% to 55% after treatment with the composition than the hair fiber thickens in diameter prior to treatment with the composition. In certain embodiments, the hair fiber thickens in diameter by 10%, 15%, 20%, 25%, 30%, 35%, 40%, 50%, 55% or more after treatment with the composition than the hair fiber thickens in diameter in untreated subjects who are comparable in health (e.g., scalp and hair health) to the treated subject. In some embodiments, the hair fiber thickens in diameter by 35% to 40%, 35% to 50%, or 40% to 55% after treatment with the composition than the hair fiber thickens in diameter in untreated subjects who are comparable in health (e.g., scalp and hair health) to the treated subject.

In another aspect, provided herein are methods for improving hair growth and thickening of the diameter of existing hair fiber of a subject (e.g., a human) using sNAG nanofibers or a composition thereof. In one embodiment, provided herein is a method for improving hair growth and thickening of the diameter of existing hair fiber, the method comprising administering to the scalp, hair or both of a subject (e.g., a human) a composition comprising sNAG nanofibers. In certain embodiments, the hair fiber thickens in diameter by 10%, 15%, 20%, 25%, 30%, 35%, 40%, 50%, 55% or more after treatment with the composition than the hair fiber thickens in diameter prior to treatment with the composition. In some embodiments, the hair fiber thickens in diameter by 35% to 40%, 35% to 50%, or 40% to 55% more after treatment with the composition than the hair fiber thickens in diameter prior to treatment with the composition. In certain embodiments, the hair fiber thickens in diameter by 10%, 15%, 20%, 25%, 30%, 35%, 40%, 50%, 55% or more after treatment with the composition than the hair fiber thickens in diameter in untreated subjects who are comparable in health (e.g., scalp and hair health) to the treated subject. In some embodiments, the hair fiber thickens in diameter by 35% to 40%, 35% to 50%, or 40% to 55% more after treatment with the composition than the hair fiber thickens in diameter in untreated subjects who are comparable in health (e.g., scalp and hair health) to the treated subject. In certain embodiments, the hair grows at a 10%, 15%, 20%, 25%, 30%, 35%, 40%, 50%, 55% or greater rate after treatment with the composition than the rate of hair growth observed prior to treatment with the composition. In some embodiments, the hair grows at a 35% to 40%, 35% to 50%, 40% to 55% greater after treatment with the composition rate than the rate of hair growth observed prior to treatment with the composition. In certain embodiments, the hair grows at a 10%, 15%, 20%, 25%, 30%, 35%, 40%, 50%, 55% or greater rate than the rate of hair growth observed in untreated subjects who are comparable in health (e.g., scalp and hair health) to the treated subject. In some embodiments, the hair grows at a 35% to 40%, 35% to 50%, or 40% to 55% greater rate than the rate of hair growth observed in untreated subjects who are comparable in health (e.g., scalp and hair health) to the treated subject.

In another aspect, provided herein are methods for reducing hair shedding using sNAG nanofibers or a composition thereof. In one embodiment, provided herein is a method for reducing hair shedding, the method comprising administering to the scalp, hair or both of a subject (e.g., a human) a composition comprising sNAG nanofibers.

In another aspect, provided herein are methods for treating hair loss using sNAG nanofibers or a composition thereof. In one embodiment, provided herein is a method for treating hair loss, the method comprising administering to the scalp, hair or both of a subject (e.g., a human) a composition comprising sNAG nanofibers.

In another aspect, provided herein are methods for promoting healthier hair of a subject (e.g., a human) using sNAG nanofibers or a composition thereof. In one embodiment, provided herein is a method for promoting healthier hair, the method comprising administering to the scalp, hair or both of a subject (e.g., a human) a composition comprising sNAG nanofibers. In a specific embodiment, the healthier hair promoted has one, two, or more, or all of the following characteristics: thicker hair, hair with improved texture, hair with greater volume, and softer hair.

In another aspect, provided herein are methods for strengthening hair follicles of a subject (e.g., a human) using sNAG nanofibers or a composition thereof. In one embodiment, provided herein is a method for strengthening hair follicles of a subject (e.g., a human), the method comprising administering to the scalp, hair or both of the subject a composition comprising sNAG nanofibers.

In another aspect, provided herein are methods for protecting the scalp of a subject (e.g., a human) against oxidative stress using sNAG nanofibers or a composition thereof. In one embodiment, provided herein is a method for protecting the scalp of a subject (e.g., a human) against oxidative stress, the method comprising administering to the scalp, hair or both of the subject a composition comprising sNAG nanofibers.

In another aspect, provided herein are methods for regenerating the scalp of a subject (e.g., a human) using sNAG nanofibers or a composition thereof. In one embodiment, provided herein is a method for regenerating the scalp of a subject (e.g., a human), the method comprising administering to the scalp, hair or both of the subject a composition comprising sNAG nanofibers. In a specific embodiment, the method results in one or more of the following: moisturizes the scalp and makes the scalp more flexible, pliable, and refreshed.

In another aspect, provided herein are methods for treating psoriasis of the scalp of a subject (e.g., a human) using sNAG nanofibers or a composition thereof. In one embodiment, provided herein is a method for treating psoriasis of the scalp of a subject (e.g., a human), the method comprising administering to the scalp, hair or both of the subject a composition comprising sNAG nanofibers. In certain embodiments, the scalp psoriasis is mild, moderate or mild to moderate.

In another aspect, provided herein are methods for treating dermatitis of the scalp of a subject (e.g., a human) using sNAG nanofibers or a composition thereof. In one embodiment, provided herein is a method for treating dermatitis of the scalp of a subject (e.g., a human), the method comprising administering to the scalp, hair or both of the subject a composition comprising sNAG nanofibers. In certain embodiments, the dermatitis is seborrheic dermatitis. In some embodiments, the dermatitis is atopic dermatitis, contact dermatitis, or dermatitis caused by a bacterial infection. In other embodiments, the dermatitis is not atopic dermatitis, contact dermatitis, or dermatitis caused by a bacterial infection.

In another aspect, provided herein are methods for treating an inflammatory condition of the scalp of a subject (e.g., a human) using sNAG nanofibers or a composition thereof. In one embodiment, provided herein is a method for treating an inflammatory condition of the scalp of a subject (e.g., a human), the method comprising administering to the scalp, hair or both of the subject a composition comprising sNAG nanofibers.

In certain embodiments, more than 50% of the sNAG nanofibers are between about 1 to 15 μm in length and the sNAG nanofibers comprise glucosamine monosaccharides, and wherein at least 70% of the monosaccharides are N-acetylglucosamine monosaccharides. In some embodiments, the sNAG nanofibers have an average length of between about 1 to 10 μm (e.g., between 1 to 8 μm or 1 to 5 μm), the sNAG nanofibers are between approximately 60,000 daltons and approximately 80,000 daltons, and the sNAG nanofibers comprise glucosamine monosaccharides, and wherein at least 70% of the monosaccharides are N-acetylglucosamine monosaccharides. See, e.g., Section 4.1, supra, and Section 5, infra, for further description of the sNAG nanofibers that may be used in the methods.

In certain embodiments, a composition described herein is administered topically to the scalp, hair or both of a subject. In a specific embodiment, a composition described herein is sprayed directly onto the scalp or a close to as possible to the scalp of a subject and massaged in. In certain embodiments, a composition described herein that is administered to the scalp, hair or both of a subject is formulated as a suspension, serum or gel. See, e.g., Section 4.3, supra, for a description of such formulations as well as other ways that composition described herein administered to the scalp, hair or both of the subject may be formulated. In certain embodiments, a composition described herein administered to the scalp, hair or both of the subject comprises the sNAG nanofibers as the sole active ingredient. In other embodiments, a composition described herein administered to the scalp, hair or both of the subject comprises one or more active ingredients, such as described in Section 4.3, supra, and Section 4.7, infra.

In a specific embodiment, an effective amount of a composition described herein comprising sNAG nanofibers is administered to the scalp, hair or both of a subject. In a specific embodiment, an effective amount of a composition described herein comprising sNAG nanofibers is sprayed directly onto the scalp or a close to as possible to the scalp of a subject and massaged in. In certain embodiments, the effective amount may achieve one, two or more, or all of the following: moisturizes the scalp, balances the scalp, protects the scalp, clears debris of old cells, unclogs hair follicles, thickens the diameter of existing hair fibers, reduces hair shedding, improves health hair, improves hair texture, improves hair volume, improves hair thickness, and improves hair softness. See, e.g., Section 4.6, infra, for amounts of sNAGs that may be administered to the scalp, hair or both of a subject. In a specific embodiment, the effective amount achieves one or more of the results noted in Section 5, infra.

In particular embodiments, treatment of the scalp, hair or both of a subject in accordance with the methods described herein achieves one, two or more of the following: moisturizes the scalp, balances the scalp, protects the scalp, clears debris of old cells, unclogs hair follicles, thickens the diameter of existing hair fibers, reduces hair shedding, improves health hair, improves hair texture, improves hair volume, improves hair thickness, and improves hair softness. In some embodiments, treatment of the scalp, hair or both of a subject in accordance with the methods described herein achieves one, two, three or more, or all of the following: (1) a moisturized, nourished and balanced scalp; (2) an increase in hair fiber diameter; (3) less scalp visisble; (4) greater hair volume; (5) less frizzy hair; (6) softer hair; (7) an increase in hair growth; (8) thicker hair; (9) healthier hair; (10) shinier hair; (11) hair with improved texture; and (12) more youthful hair.

In some embodiments, approximately 7 to approximately 15 days after a subject has applied a composition described herein to their scalp, or both their scalp and hair, the subject may notice one, two, three or all of the following: (1) less hair in the shower drain, on their hairbrush, on their pillow and clothes, or on their hands when conditioning hair; (2) hair with more shine and hydration; (3) less frizzy hair; and (4) softer hair. In certain embodiments, approximately 20 to approximately 25 days after a subject has applied a composition described herein to their scalp, or both their scalp and hair, the subject may notice one, two, three or all of the following: (1) hair with more volume (2) thicker hair; (3) more youthful hair; (4) healthier hair; (5) plumper hair; and (6) hair with a more pleasing shape. In some embodiments, approximately 30 to approximately 50 days after a subject has applied a composition described herein to their scalp, or both their scalp and hair, the subject may notice one, two, three or all of the following: (1) stronger hair; (2) healthier hair; (3) hair with better texture; (4) shinier hair; (5) increased hair density; (6) less visible scalp; (7) thicker hair; and (8) hair with more volume.

In certain embodiments, a composition described herein is administered to the scalp, hair or both of a subject alone or in combination with another therapy. See, e.g., Section 4.7 for therapies or agents that may be administered to a subject in conjunction with a composition described herein. In a specific embodiment, a composition described herein is administered alone to the scalp, hair or both a subject.

In another aspect, provided herein are compositions comprising glucosamine (e.g. D-(+)-glucosamine hydrochloride, N-acetyl glucosamine, glucosamine sulfate) for use in any of the methods described herein, wherein the composition does not include sNAG nanofibers. In another aspect, provided herein are compositions comprising glucosamine (e.g. D-(+)-glucosamine hydrochloride, N-acetyl glucosamine, glucosamine sulfate) for use in any of the methods described herein, wherein the composition does not include chitosan or chitin. In another aspect, provided herein are compositions comprising glucosamine (e.g. D-(+)-glucosamine hydrochloride, N-acetyl glucosamine, glucosamine sulfate) for use in any of the methods described herein, wherein the composition does not include sNAG nanofibers, chitin or chitosan. In a specific embodiment, the glucosamine is the only active ingredient in the composition. In some embodiments, the composition comprises one or more preservatives (e.g., phenoxyethanol, caprylyl glycol, phenethyl alcohol, pentylene glycol or propanediol). In a specific embodiment, the glucosamine composition described in Section 5.4 is used in any of the methods described herein.

4.5 Patient Populations

In some embodiments, a subject being treated in accordance with the methods described herein is an animal. In certain embodiments, the animal is a canine. In certain embodiments, the animal is a horse. In certain embodiments, the animal is a cow. In certain embodiments, the animal is a mammal, e.g., a horse, swine, or primate, preferably a human. In some embodiments, the animal is a pet or a farm animal. In a preferred embodiment, a subject being treated in accordance with the methods described herein is a human. In another preferred embodiment, a subject being treated in accordance with the methods described herein is a healthy human or a human with a healthy scalp.

In certain embodiments, a subject being treated in accordance with the methods described herein is a human adult. In certain embodiments, a subject being treated in accordance with the methods described herein is a human adult more than 50 years old. In certain embodiments, a subject being treated in accordance with the methods described herein is an elderly human subject. In certain embodiments, a subject being treated in accordance with the methods described herein is a human child. In some embodiments, a subject being treated in accordance with the methods described herein is a human female (in specific embodiments, a healthy human female). In other embodiments, a subject being treated in accordance with the methods described herein is human male (in specific embodiments, a healthy human male).

In certain embodiments, a subject being treated in accordance with the methods described herein has either thinning hair, loss of hair volume, or both. In some embodiments, a subject being treated in accordance with the methods described herein has a hair count of 10, 11, 12, 13, 14, 15, 16, 17, 18, 19, 20, 21, 22 or more in a Hair Pull Test, such as known in the art or described herein. In certain embodiments, a subject being treated in accordance with the methods described herein has a hair count of 10 to 15, 15 to 20, 10 to 20, or 15 to 30 in a Hair Pull Test, such as known in the art or described herein.

In certain embodiments, a subject being treated in accordance with the methods described herein is experiencing patterned baldness. In a specific embodiment, the subject is experiencing patterned baldness on the Hamilton-Norwood scale of type 1, 2, 3, 4, 5, 6 or 7. In a specific embodiment, the subject is experiencing patterned baldness on the Hamilton-Norwood scale of type 1 to 5, 1 to 4, 1 to 3, or 1 to 2.

In some embodiments, a subject being treated in accordance with the methods described herein is not experiencing patterned baldness. In a specific embodiment, the subject is not experiencing patterned baldness on the Hamilton-Norwood scale of type 1, 2, 3, 4, 5, 6 or 7.

In certain embodiments, a subject being treated in accordance with the methods described herein has Type I on the Fitzpatrick Scale. In some embodiments, a subject being treated in accordance with the methods described herein has Type II on the Fitzpatrick Scale. In certain embodiments, a subject being treated in accordance with the methods described herein has Type III on the Fitzpatrick Scale. In some embodiments, a subject being treated in accordance with the methods described herein has Type IV on the Fitzpatrick Scale. In certain embodiments, a subject being treated in accordance with the methods described herein has Type V on the Fitzpatrick Scale. In some embodiments, a subject being treated in accordance with the methods described herein has Type VI on the Fitzpatrick Scale.

In a specific embodiment, a subject being treated in accordance with the methods described herein has a concern about one, two, or more, or all of the following: hair shedding, hair loss, hair thickness, hair volume and overall hair health.

In certain embodiments, a subject being treated in accordance with the methods described herein has not been not diagnosed with a bacterial, fungal, parasitic or viral infection. In particular embodiments, a subject being treated in accordance with the methods described herein has not been not diagnosed with a bacterial, fungal, parasitic or viral infection affecting the scalp, hair or both. In certain embodiments, a subject being treated in accordance with the methods described herein has a bacterial, fungal, parasitic or viral infection, which does not affect the scalp, hair or both. In some embodiments, a subject being treated in accordance with the methods described herein does not display one or more symptoms of a bacterial, fungal, parasitic or viral infection. In particular embodiments, a subject being treated in accordance with the methods described herein does not display one or more symptoms of a bacterial, fungal, parasitic or viral infection affecting the scalp, hair or both.

In certain embodiments, a subject being treated in accordance with the methods described herein does not have a malassezia infection. In other embodiments, a subject being treated in accordance with the methods described herein has a malassezia infection. In some embodiments, a subject being treated in accordance with the methods described herein does not have a malassezia infection affecting the scalp, hair or both. In certain embodiments, a subject being treated in accordance with the methods described herein has a malassezia infection, which does not affect their scalp, hair or both.

In some embodiments, a subject being treated in accordance with the methods described herein has dermatitis. In particular embodiments, a subject being treated in accordance with the methods described herein has atopic dermatitis. In some embodiments, a subject being treated in accordance with the methods described herein has seborrheic dermatitis. In certain embodiments, a subject being treated in accordance with the methods described herein has dermatitis caused by a bacterial infection. In certain embodiments, a subject being treated in accordance with the methods described herein has contact dermatitis.

In some embodiments, a subject being treated in accordance with the methods described herein has dermatitis affecting their scalp. In particular embodiments, a subject being treated in accordance with the methods described herein has atopic dermatitis affecting the scalp. In some embodiments, a subject being treated in accordance with the methods described herein has seborrheic dermatitis affecting the scalp. In certain embodiments, a subject being treated in accordance with the methods described herein has dermatitis affecting the scalp, which is caused by a bacterial infection. In some embodiments, a subject being treated in accordance with the methods described herein has contact dermatitis affecting the scalp.

In certain embodiments, a subject being treated in accordance with the methods described herein does not have scalp inflammation. In other embodiments, a subject being treated in accordance with the methods described herein has scalp inflammation.

In certain embodiments, a subject being treated in accordance with the methods described herein does not have a condition that causes scalp inflammation. In some embodiments, a subject being treated in accordance with the methods described herein has not been diagnosed with a condition that causes scalp inflammation.

In certain embodiments, a subject being treated in accordance with the methods described herein has a condition that causes scalp inflammation. In some embodiments, a subject being treated in accordance with the methods described herein has been diagnosed with a condition that causes scalp inflammation. In certain embodments, a subject being treated in accordance with the methods described herein has a condition that causes scalp inflammation but is not experiencing any symptoms of the condition during all or part of the treatment. In some embodments, a subject being treated in accordance with the methods described herein has a condition that causes scalp inflammation but is not experiencing any scalp inflammation during all or part of the treatment.

In some embodiments, a subject being treated in accordance with the methods described herein has dermatitis, which is not affecting their scalp. In particular embodiments, a subject being treated in accordance with the methods described herein has atopic dermatitis, which is not affecting the scalp. In some embodiments, a subject being treated in accordance with the methods described herein has seborrheic dermatitis, which is not affecting the scalp. In certain embodiments, a subject being treated in accordance with the methods described herein has dermatitis caused by a bacterial infection, which is not affecting their scalp. In some embodiments, a subject being treated in accordance with the methods described herein has contact dermatitis, which is not affecting the scalp

In some embodiments, a subject being treated in accordance with the methods described herein does not have dermatitis. In particular embodiments, a subject being treated in accordance with the methods described herein does not have atopic dermatitis. In some embodiments, a subject being treated in accordance with the methods described herein does not have seborrheic dermatitis. In certain embodiments, a subject being treated in accordance with the methods described herein does not have dermatitis caused by a bacterial infection. In some embodiments, a subject being treated in accordance with the methods described herein does not have contact dermatitis.

In some embodiments, a subject being treated in accordance with the methods described herein does not psoriasis. In certain embodiments, a subject being treated in accordance with the methods described herein does not have scalp psoriasis. In some embodiments, a subject being treated in accordance with the methods described herein has psoriasis, which does not affect the scalp.

In some embodiments, a subject being treated in accordance with the methods described herein has psoriasis. In certain embodiments, a subject being treated in accordance with the methods described herein has scalp psoriasis. In specific embodiments, a subject being treated in accordance with the methods described herein has mild, moderate, or mild to moderate scalp psoriasis.

In certain embodiments, the scalp of a subject being treated in accordance with the methods described herein does not have wound (e.g., an open wound). In other embodiments, the scalp of a subject being treated in accordance with the methods described herein has a wound (e.g., an open wound). In certain embodiments, a subject being treated in accordance with the methods described herein has a wound but not on their scalp.

In certain embodiments, a subject being treated in accordance with the methods described herein does not have tinea captitis. In other embodiments, a subject being treated in accordance with the methods described herein has tinea captitis.

In certain embodiments, a subject being treated in accordance with the methods described herein does not have chronic skin allergies. In other embodiments, a subject being treated in accordance with the methods described herein has chronic skin allergies.

In certain embodiments, a subject being treated in accordance with the methods described herein does not have folliculitis. In other embodiments, a subject being treated in accordance with the methods described herein has folliculitis.

In certain embodiments, a subject being treated in accordance with the methods described herein does not a hair loss condition. In some embodiments, the subject being treated in accordance with the methods described herein does not have hair loss caused by or associated with medication, such as chemotherapy (e.g., anti-cancer therapy or cytotoxic drugs), thallium compounds, vitamins (e.g., vitamin A), retinoids, anti-viral therapy, or psychological therapy, or radiation (such as the banding pattern of scalp hair loss that may be caused by radiation overdose). In certain embodiments, the subject being treated in accordance with the methods described herein does not have hair loss caused by or associated with one, two or more, or any of the following: trauma, endocrine dysfunction, surgery, physical trauma, x-ray atrophy, burning or other injury or wound, stress, aging, an autoimmune disease or disorder, malnutrition, an infection (such as, e.g., a fungal, viral, or bacterial infection, including chronic deep bacterial or fungal infections), dermatitis, psoriasis, eczema, pregnancy, allergy, a severe illness (e.g., scarlet fever), myxedema, hypopituitarism, early syphilis, discoid lupus erythematosus, cutaneous lupus erythematosus, lichen planus, deep factitial ulcer, granuloma (e.g., sarcoidosis, syphilitic gummas, TB), inflamed tinea capitis (kerion, favus), a slow-growing tumor of the scalp or other skin tumor, or any other disease or disorder associated with or that causes balding or hair loss known in the art.

In some embodiments, a subject being treated in accordance with the methods described herein does not have a condition characterized as diffuse hair loss, such as Telogen effluvium, female pattern hair loss (FPHL), male pattern hair loss (MPHL; a type of “androgenetic alopecia”), chronic TE, anagen effluvium, loose anagen hair syndrome, diffuse type of alopecia areata, congenital atrichia, congenital hypotrichosis and hair shaft abnormalities (hair breakage, unruly hair).

In specific embodiments, a subject being treated in accordance with the methods described herein has not been diagnosed as having any form of baldness or alopecia. In some embodiments, a subject being treated in accordance with the methods described herein does not have one, two, or more, or any of the following conditions: alopecia areata, cyclic alopecia, loose anagen syndrome, acute anagen, and trichotillomania.

In certain embodiments, a subject being treated in accordance with the methods described herein has a hair loss condition. In some embodiments, the subject being treated in accordance with the methods described herein has hair loss caused by or associated with medication, such as chemotherapy (e.g., anti-cancer therapy or cytotoxic drugs), thallium compounds, vitamins (e.g., vitamin A), retinoids, anti-viral therapy, or psychological therapy, or radiation (such as the banding pattern of scalp hair loss that may be caused by radiation overdose). In a specific embodiment, a subject treated in accordance with the methods described herein has hair loss caused by or associated with chemotherapy. In certain embodiments, the subject being treated in accordance with the methods described herein has hair loss caused by or associated with trauma, endocrine dysfunction, surgery, physical trauma, x-ray atrophy, burning or other injury or wound, stress, aging, an autoimmune disease or disorder, malnutrition, an infection (such as, e.g., a fungal, viral, or bacterial infection, including chronic deep bacterial or fungal infections), dermatitis, psoriasis, eczema, pregnancy, allergy, a severe illness (e.g., scarlet fever), myxedema, hypopituitarism, early syphilis, discoid lupus erythematosus, cutaneous lupus erythematosus, lichen planus, deep factitial ulcer, granuloma (e.g., sarcoidosis, syphilitic gummas, TB), inflamed tinea capitis (kerion, favus), a slow-growing tumor of the scalp or other skin tumor, or any other disease or disorder associated with or that causes balding or hair loss known in the art.

In some embodiments, a subject being treated in accordance with the methods described herein has a condition characterized as diffuse hair loss, such as Telogen effluvium, female pattern hair loss (FPHL), male pattern hair loss (MPHL; a type of “androgenetic alopecia”), chronic TE, anagen effluvium, loose anagen hair syndrome, diffuse type of alopecia areata, congenital atrichia, congenital hypotrichosis and hair shaft abnormalities (hair breakage, unruly hair).

In certain embodiments, a subject being treated in accordance with the methods described herein has been diagnosed as having a form of baldness or alopecia. In some embodiments, a subject being treated in accordance with the methods described herein has one, two, or more, or all of the following conditions: alopecia areata, cyclic alopecia, loose anagen syndrome, acute anagen, and trichotillomania.

In certain embodiments, a subject being treated in accordance with the methods described herein has scarring alopecia (e.g., primary cicatricial alopecia (PCA) and secondary cicatricial alopecia). In other embodiments, a subject being treated in accordance with the methods described herein does not have scarring alopecia (e.g., primary cicatricial alopecia (PCA) and secondary cicatricial alopecia). Primary cicatricial alopecias include lymphocyte-mediated PCAs, such as lichen planopilaris (LPP), frontal fibrosing alopecia (FFA), central centrifugal cicatricial alopecia (CCCA), and pseudopelade (Brocq); neutrophil-mediated PCAs, such as folliculitis decalvans and tufted folliculitis; and PCAs involving a mixed inflammatory infiltrate, such as occurs in dissecting cellulitis and folliculitis keloidalis.

In certain embodiments, a subject being treated in accordance with the methods described herein has nonscarring alopecia (e.g., focal alopecia). In other embodiments, a subject being treated in accordance with the methods described herein does not have nonscarring alopecia.

In certain embodiments, a subject being treated in accordance with the methods described herein has androgenetic alopecia. In other embodiments, a subject being treated in accordance with the methods described herein does not have androgenetic alopecia.

In certain embodiments, a subject being treated in accordance with the methods described herein has male or female pattern hair loss. In other embodiments, a subject being treated in accordance with the methods described herein does not have male or female pattern hair loss.

In certain embodiments, a subject being treated in accordance with the methods described herein has diffuse alopecia areata. In other embodiments, a subject being treated in accordance with the methods described herein does not have diffuse alopecia areata.

In certain embodiments, a subject being treated in accordance with the methods described herein has telogen effluvium. In other embodiments, a subject being treated in accordance with the methods described herein does not have telogen effluvium.

In certain embodiments, a subject being treated in accordance with the methods described herein has anagen effluvium. In other embodiments, a subject being treated in accordance with the methods described herein does not have anagen effluvium.

In certain embodiments, a subject being treated in accordance with the methods described herein has alopecia totalis. In other embodiments, a subject being treated in accordance with the methods described herein does not have alopecia totalis.

In certain embodiments, a subject being treated in accordance with the methods described herein has alopecia universalis. In other embodiments, a subject being treated in accordance with the methods described herein does not have alopecia universalis.

In certain embodiments, a subject being treated in accordance with the methods described herein has traction alopecia. In other embodiments, a subject being treated in accordance with the methods described herein does not have traction alopecia.

In certain embodiments, a subject being treated in accordance with the methods described herein has cancer. In some embodiments, a subject being treated in accordance with the methods described herein has been diagnosed with cancer. In certain embodiments, a subject being treated in accordance with the methods described herein has undergone treatment for cancer. In specific embodiments, a subject being treated in accordance with the methods described herein has undergone treatment for cancer that causes or has caused hair loss.

In certain embodiments, a subject being treated in accordance with the methods described herein does not have cancer. In some embodiments, a subject being treated in accordance with the methods described herein has not been diagnosed with cancer. In certain embodiments, the subject being treated in accordance with the methods described herein has not undergone treatment for cancer. In specific embodiments, a subject being treated in accordance with the methods described herein has not undergone treatment for cancer that causes or has caused hair loss.

In certain embodiments, a subject being treated in accordance with the methods described herein does not have an autoimmune condition (e.g., vitiligo, diabetes, thyroid disease, rheumatoid arthritis, or discoid lup erythematosus). In some embodiments, a subject being treated in accordance with the methods described herein has not been diagnosed with an autoimmune condition (e.g., vitiligo, diabetes, thyroid disease, rheumatoid arthritis, or discoid lup erythematosus).

In certain embodiments, a subject being treated in accordance with the methods described herein has an autoimmune condition (e.g., vitiligo, diabetes, thyroid disease, rheumatoid arthritis, or discoid lup erythematosus). In some embodiments, a subject being treated in accordance with the methods described herein has been diagnosed with an autoimmune condition (e.g., vitiligo, diabetes, thyroid disease, rheumatoid arthritis, or discoid lup erythematosus).

In certain embodiments, a subject being treated in accordance with the methods described herein has not used any topical or systemic hair loss, or hair growth treatments 2 weeks, 3 weeks, 1 month, 2 months, 3 months, 5 months, or 6 months prior to initiation of a method of treatment described herein. In some embodiments, a subject being treated in accordance with the methods described herein has not used any topical or systemic hair loss, or hair growth treatments 1 to 3 months, 1 to 6 months, or 3 to 6 months prior to initiation of a method of treatment described herein. In certain embodiments, a subject being treated in accordance with the methods described herein has not used any topical or systemic hair loss, or hair growth treatments 1 year prior to initiation of a method of treatment described herein. In some embodiments, a subject being treated in accordance with the methods described herein has not used any topical or systemic hair loss, or hair growth treatments or has no recollection of using any topical or systemic hair loss or hair growth treatments.

In certain embodiments, a subject being treated in accordance with the methods described herein has not used either Rogaine, Minoxidil or both 2 weeks, 3 weeks, 1 month, 2 months, 3 months, 5 months, or 6 months prior to initiation of a method of treatment described herein. In some embodiments, a subject being treated in accordance with the methods described herein has not used either Rogaine, Minoxidil or both 1 to 3 months, 1 to 6 months, or 3 to 6 months prior to initiation of a method of treatment described herein. In certain embodiments, a subject being treated in accordance with the methods described herein has not used either Rogaine, Minoxidil or both 1 year prior to initiation of a method of treatment described herein. In some embodiments, a subject being treated in accordance with the methods described herein has not used either Rogaine, Minoxidil or both or has no recollection of using either Rogaine, Minoxidil or both. In certain embodiments, a subject being treated in accordance with the methods described herein has not used a nutritional supplement.

In certain embodiments, a subject being treated in accordance with the methods described herein has not used a composition containing minoxidil 2 weeks, 3 weeks, 1 month, 2 months, 3 months, 5 months, or 6 months prior to initiation of a method of treatment described herein. In some embodiments, a subject being treated in accordance with the methods described herein has not used a composition containing minoxidil 1 to 3 months, 1 to 6 months, or 3 to 6 months prior to initiation of a method of treatment described herein. In certain embodiments, a subject being treated in accordance with the methods described herein has not used a composition containing minoxidil 1 year prior to initiation of a method of treatment described herein. In some embodiments, a subject being treated in accordance with the methods described herein has not used a composition containing minoxidil or has no recollection of using a composition containing minoxidil.

In specific embodiments, a subject being treated in accordance with the methods described herein has not used any one, two or more, or all of the following: Rogaine, Minoxidil, Keranique®, steroids (oral or topical), corticosteroids, anthralin cream, spironolactone, flutamide, ketoconazole, and pyrithione zinc. In some embodiments, a subject being treated in accordance with the methods described herein has not used immunotherapy. In some embodiments, a subject being treated in accordance with the methods described herein has not used any one, two or more of the following: terbinafine, fluconazole, itraconazole, or griseofulvin.

In certain embodiments, a subject being treated in accordance with the methods described herein has used any topical or systemic hair loss, or hair growth treatments. In some embodiments, a subject being treated in accordance with the methods described herein has used either Rogaine, Minoxidil or both. In certain embodiments, a subject being treated in accordance with the methods described herein has used a nutritional supplement.

In specific embodiments, a subject being treated in accordance with the methods described herein has used any one, two or more, or all of the following: Rogaine, Minoxidil, steroids (oral or topical), corticosteroids, anthralin cream, spironolactone, flutamide, ketoconazole, and pyrithione zinc. In some embodiments, a subject being treated in accordance with the methods described herein has used immunotherapy. In some embodiments, a subject being treated in accordance with the methods described herein has used any one, two or more of the following: terbinafine, fluconazole, itraconazole, or griseofulvin.

In specific embodiments, a subject being treated in accordance with the methods described herein meets one, two, or more, or all of the inclusion criteria identified in an example in Section 5, infra. In specific embodiments, a subject being treated in accordance with the methods described herein does not meet one, two, or more, or all of the exclusion criteria identified in an example in Section 5, infra. In specific embodiments, a subject being treated in accordance with the methods described herein meets one, two, or more, or all of the inclusion criteria identified in an example in Section 5, infra, and does not meet one, two, or more, or all the exclusion criteria identified in an example in Section 5, infra.

4.6 Modes of Administration of sNAG Nanofiber Compositions

The sNAG nanofibers or a sNAG nanofiber composition described herein may be applied topically to a surface of the scalp, hair or both of a patient. In a specific embodiment, the sNAG nanofibers or a composition thereof is sprayed onto a subject's scalp, hair or both. In certain embodiments, the scalp, hair or both is sprayed multiple times in one sitting with the sNAG nanofibers or a composition thereof. For example, a composition described herein may be applied (e.g., sprayed) onto the scalp of a subject or as close to the scalp of a subject as possible and the composition massaged in. The hair of a subject may be parted in order to apply (e.g., spray) a composition described herein directly to the scalp of a subject or as close to the scalp of a subject as possible.

The above-listed methods for administration may include administration of the sNAG nanofiber or a composition thereof in the form of suspension, a gel, a serum, a liquid solution, a spray, or any other formulation described herein or known in the art. In a specific embodiment, the composition for administration is formulated as a liquid formulation. In a specific embodiment, the composition for administration is formulated as a suspension, a serum or gel. In certain embodiments, sNAG nanofibers or a composition thereof are applied to the scalp, hair or both of a subject. In some embodiments, the hair of the subject is wet. In other embodiments, the hair of the subject is dry. For example, a composition described herein may be applied (e.g., sprayed) onto the scalp of a subject or as close to the scalp of a subject as possible in the morning after shampooing and conditioning their hair and left in on their damp scalp. In another example, a composition described herein is applied (e.g., sprayed) onto the scalp of a subject or as close to the scalp of a subject as possible on a dry scalp at night before bed (e.g., an hour before bed) and left in.

In certain embodiments, sNAG nanofibers or a composition thereof are applied to the scalp, hair or both of dried hair of a subject. In some embodiments, sNAG nanofibers or a composition thereof are applied to the scalp, hair or both of dried hair of a subject after shampooing, conditioning, or both. In certain embodiments, sNAG nanofibers or a composition thereof are applied to the scalp, hair or both of wet hair of a subject. In some embodiments, sNAG nanofibers or a composition thereof are applied on wet hair of a subject and left in. The hair may then be dried and styled. In a specific embodiment, sNAG nanofibers or a composition thereof is left in the scalp, hair or both after application. In specific embodiments, sNAG nanofibers or a composition thereof are not washed out of the scalp, hair or both after application. In specific embodiments, sNAG nanofibers or a composition thereof are left on the scalp, hair or both after application until the hair is washed the next time. In certain embodiments, sNAG nanofibers or a composition thereof are/is applied to the scalp, hair or both and the hair is not washed and the sNAG nanofibers or composition are/is not otherwise removed for a period of at least 1 hour, 2 hours, 3 hours, 4 hours, 5 hours, 6 hours, 9 hours, 12 hours, 18 hours or 24 hours after application. In some embodiments, sNAG nanofibers or a composition thereof are/is applied to the scalp, hair or both and the hair is not washed and the sNAG nanofibers or composition are/is not otherwise removed for a period of at least 1 to 2 hours, 3 to 6 hours, 3 to 9 hours, 6 to 9 hours, 9 to 12 hours, 6 to 12 hours, 12 to 18 hours, 18 to 24 hours or 24 to 48 hours after application.

In certain embodiments, sNAG nanofibers or a composition thereof are applied to the scalp, hair or both of a subject before they color their hair. In some embodiments, sNAG nanofibers or a composition thereof are applied to the scalp, hair or both of a subject after they color their hair. In certain embodiments, sNAG nanofibers or a composition thereof are applied to the scalp, hair or both of a subject before and after coloring their hair.

In some embodiments, sNAG nanofibers or a composition thereof are applied (e.g., sprayed) on the over the entire scalp or a close to the scalp as possible, and the sNAG nanofibers or composition is massaged into the scalp for a period of time (e.g., for approximately 10-15 seconds, approximately 10-30 seconds, approximately 30 to 60 second, approximately 30 to 90 seconds, approximately 1 to 3 minutes, or approximately 1 to 5 minutes). In certain embodiments, sNAG nanofibers or a composition thereof are applied (e.g., sprayed) on the over the entire scalp and hair, and the sNAG nanofibers or composition is massaged into the hair and scalp for a period of time (e.g., for approximately 10-15 seconds, approximately 10-30 seconds, approximately 30 to 60 second, approximately 30 to 90 seconds, approximately 1 to 3 minutes, or approximately 1 to 5 minutes). In a specific embodiment, sNAG nanofibers or a composition thereof are applied (e.g., sprayed) as described in Section 5, infra (in particular, Section 5.1 to 5.3, 5.5 and 5.6 infra).

In a specific embodiment, sNAG nanofibers or a composition thereof are applied (e.g., sprayed) to parted hair at the roots, as close to the scalp as possible, several times, until the scalp is covered with sNAG nanofibers or composition. In specific embodiments, the sNAG nanofibers or composition is thoroughly massaged on to the scalp for 25-30 seconds.

Contemplated treatment regimens include a regiment of multiple doses or multiple applications of sNAG nanofibers or a sNAG nanofiber composition. A dose or an application may be administered, e.g., daily, every other day, weekly or monthly. For example, a dose of sNAG nanofibers or a composition thereof may be administered every 24 hours, every 48 hours, every 72 hours, once a week, 2 times a week, 3 times a week, every other day, or once in 2 weeks. See, e.g., Section 5, infra (in particular Sections 5.1 to 5.3, 5.5, and 5.6, infra) regarding dosing of sNAG nanofibers to subjects.

sNAG nanofibers or a sNAG nanofiber composition may be administered for a duration equal to or greater than 1 week, 2 weeks, 3 weeks, 1 month, 2 months, 3 months, 4 months, 5 months, 6 months, 7 months, 8 months, 9 months, 10 months, 11 months, 1 year, 1.5 years, 2 years, 2.5 years, 3 years, 4 years, 5 years, 7 years, 10 years or more. In a specific embodiment, sNAG nanofibers or a sNAG nanofiber composition are administered for as long as a subject desires. In certain embodiments, sNAG nanofibers or a sNAG nanofiber composition is administered to a subject until a subject is satisfied with one, two, or more or all of the following: the overall health of their hair, the health of their scalp, the length of their hair, the thickness of their hair, the volume of their hair, the shine of their hair, and the diameter of their hair fibers.

In one embodiment, sNAG nanofibers or a sNAG nanofiber composition does not cause any side effects or causes only mild side effects during the duration of the treatment. In another embodiment, sNAG nanofibers or a sNAG nanofiber composition does not cause irritation (e.g., moderate or severe irritation) or allergy (e.g., moderate or severe allergy).

In certain embodiments, sNAG nanofibers or a sNAG nanofiber composition are administered once per day for a period of approximately 1 month to approximately 6 months, approximately 1 month to approximately 5 months, approximately 1 month to approximately 4 months, approximately 1 month to approximately 3 months, or approximately 1 month to approximately 2 months. In some embodiments, sNAG nanofibers or a sNAG nanofiber composition are administered once per day for a period of approximately 2 months to approximately 6 months, approximately 2 month to approximately 5 months, approximately 2 month to approximately 4 months, or approximately 2 month to approximately 3 months. In certain embodiments, sNAG nanofibers or a sNAG nanofiber composition are administered once per day for a period of approximately 3 months to approximately 6 months, approximately 3 month to approximately 5 months, or approximately 3 month to approximately 4 months. In a specific embodiment, sNAG nanofibers or a sNAG nanofiber composition are administered once per day for a period of approximately 1 month to approximately 3 months. In another specific embodiment, sNAG nanofibers or a sNAG nanofiber composition are administered once per day for a period of approximately 3 months.

In certain embodiments, after an initial treatment period with sNAG nanofibers or a sNAG nanofiber composition, then a maintenance administration may be used. The maintenance administration may be 1 to 5 times per week, 1 to 4 times per week, or 1 to 3 times per week. The maintenance administration may last 6 months to 1 year, 1 to 2 years, 1 to 3 years, 2 to 4 years or longer. The initial treatment period may involve once daily administration of the sNAG nanofibers or a sNAG nanofiber composition for a period of 3 months to 6 months or 3 to 9 months, 6 to 9 months, or 6 to 12 months.

In certain embodiments, sNAG nanofibers or a sNAG nanofiber composition are administered once per day for a period of approximately 1 month to approximately 6 months, approximately 1 month to approximately 5 months, approximately 1 month to approximately 4 months, approximately 1 month to approximately 3 months, or approximately 1 month to approximately 2 months, followed by a maintenance administration 1 to 5 times per week, 1 to 4 times per week, or 1 to 3 times per week. In some embodiments, sNAG nanofibers or a sNAG nanofiber composition are administered once per day for a period of approximately 2 months to approximately 6 months, approximately 2 month to approximately 5 months, approximately 2 month to approximately 4 months, or approximately 2 month to approximately 3 months, followed by a maintenance administration 1 to 5 times per week, 1 to 4 times per week, or 1 to 3 times per week. In certain embodiments, sNAG nanofibers or a sNAG nanofiber composition are administered once per day for a period of approximately 3 months to approximately 6 months, approximately 3 month to approximately 5 months, or approximately 3 month to approximately 4 months, followed by a maintenance administration 1 to 5 times per week, 1 to 4 times per week, or 1 to 3 times per week. In a specific embodiment, sNAG nanofibers or a sNAG nanofiber composition are administered once per day for a period of approximately 1 month to approximately 3 months, followed by a maintenance administration 1 to 5 times per week, 1 to 4 times per week, or 1 to 3 times per week. In another specific embodiment, sNAG nanofibers or a sNAG nanofiber composition are administered once per day for a period of approximately 3 months, followed by a maintenance administration 1 to 3 times per week.

In another specific embodiment, sNAG nanofibers or a sNAG nanofiber composition are administered once per day for a period of approximately 6 to approximately 9 months, approximately 6 to approximately 12 months, approximately 9 to approximately 12 months, approximately 12 to approximately 18 months, or approximately 18 to approximately 24 months, followed by a maintenance administration 1 to 3 times per week. In another specific embodiment, sNAG nanofibers or a sNAG nanofiber composition are administered once per day for a period of approximately 6 months, approximately 9 months, approximately 12 months, approximately 18 months, approximately 24 months, or more, followed by a maintenance administration 1 to 3 times per week.

In another specific embodiment, sNAG nanofibers or a sNAG nanofiber composition are administered once per day for a period of approximately 90 days, followed by a maintenance administration of two to three times per week. In some embodiments, sNAG nanofibers or composition thereof are used once a day for the first 3 months, then 1-3 times a week for maintenance.

In certain embodiments, a composition described herein is applied (e.g., sprayed) directly on the scalp of a subject or as close to the scalp of a subject as possible once daily for approximately 1 to 3 approximately months. In some embodiments, a composition described herein is applied (e.g., sprayed) directly on the scalp of a subject or as close to the scalp of a subject as possible once daily for approximately 3 to 6 approximately months. In certain embodiments, a composition described herein is applied (e.g., sprayed) directly on the scalp of a subject or as close to the scalp of a subject as possible once daily for approximately 6 to 9 approximately months. In some embodiments, a composition described herein is applied (e.g., sprayed) directly on the scalp of a subject or as close to the scalp of a subject as possible once daily for approximately 9 to 12 approximately months. In certain embodiments, a composition described herein is applied (e.g., sprayed) directly on the scalp of a subject or as close to the scalp of a subject as possible once daily for approximately 6 to 12 approximately months. In some embodiments, after applying (e.g, spraying) a composition described herein daily for a period of time (e.g., approximately 1 to approximately 3 months, approximately 3 to 6 months, approximately 6 to approximately 9 months, approximately 9 to approximately 12 months, or approximately 6 to approximately 12 months), a composition described herein is applied (e.g., sprayed) onto the scalp or as close as possible to the scalp 1 to 3 times per week.

Concentration of the sNAG nanofiber in a composition may vary. In general, an effective amount of the sNAG nanofiber is used. An effective amount may be an amount sufficient to achieve one or more of the effects described herein. For example, a composition may comprise about 0.05 to 5 mg of the sNAG nanofibers per mL of an isotonic solution or water in a form suitable for administration to a patient. In certain embodiments, a composition described herein comprises about 0.05 to 3 mg of the sNAG nanofibers per mL of an isotonic solution or water, about 0.5 to 5 mg of the sNAG nanofibers per mL of an isotonic solution or water, about 0.5 to 3 mg of the sNAG nanofibers per mL of an isotonic solution or water, or about 1 to 3 mg of the sNAG nanofibers per mL of an isotonic solution or water. In specific embodiments, a composition described herein comprises about 0.5 to 2 mg of the sNAG nanofibers per mL of an isotonic solution or water, about 0.5 to 1 mg of the sNAG nanofibers per mL of an isotonic solution or water, about 1 to 2 mg of the sNAG nanofibers per mL of isotonic solution or water. In other embodiments, the concentration of sNAG nanofibers in the composition is about 0.05-50 mg/mL. In other embodiments, the concentration of sNAG nanofibers in the composition is about 0.05-30 mg/mL. In other embodiments, the concentration of sNAG nanofibers in the composition is about 0.05-20 mg/mL. In other embodiments, the concentration of sNAG nanofibers in the composition is about 0.05 to 10 mg/mL. In a specific embodiment, the sNAG nanofiber concentration in a composition is the concentration set forth in Section 5.1, 5.2, 5.3, 5.5 or 5.6.

In particular embodiments, a dose of 500 microliters to 3 mL of a composition described herein is applied to the scalp, hair or both of a subject. In a specific embodiment, a dose of 800 microliters to 3 mL of a composition described herein is applied to the scalp, hair or both of a subject. In another specific embodiment, a dose of 1 mL to 3 mL of a composition described herein is applied to the scalp, hair or both of a subject. In another specific embodiment, a dose of 2 mL to 3 mL of a composition described herein is applied to the scalp, hair or both of a subject. In a specific embodiment, a dose of 800 microliters to 2 mL of a composition described herein is applied to the scalp, hair or both of a subject. In some embodiments, a dose is applied to the scalp, hair or both of a subject daily. In a specific embodiment, the composition is sprayed onto the scalp, hair or both. In some embodiments, 0.5 to 3 mg, 0.5 to 2 mg, 0.5 to 1 mg, 1 to 3 mg, 2 to 3 mg, 0.8 to 1 mg, 0.8 to 2 mg, or 0.8 to 1 mg of sNAG nanofibers are applied to the scalp, hair or both of a subject daily. In a specific embodiment, the composition is sprayed onto the scalp, hair or both.

In particular embodiments, a dose of 50 to 1,000 microliters of a composition described herein is administered to the scalp, hair or both of a subject. In a specific embodiment, a dose of 100 to 500 microliters of a composition described herein is administered to the scalp, hair or both of a subject. In another specific embodiment, a dose of 100 to 400 microliters of a composition described herein is administered to the scalp, hair or both of a subject. In another specific embodiment, a dose of 100 to 250 microliters of a composition described herein is administered to the scalp, hair or both of a subject. In another specific embodiment, a dose of 50 to 100 microliters of a composition described herein is administered to the scalp, hair or both of a subject. In some embodiments, a dose is applied to the scalp, hair or both of a subject daily. In a particular embodiment, the composition is an isotonic serum, isotonic suspension or an isotonic gel. In certain embodiments, 0.005 to 2 mg, 0.005 to 1 mg, 0.005 to 0.5 mg, 0.005 to 0.2 mg, 0.5 to 2 mg, 0.5 to 1 mg, 0.1 to 2 mg, 0.15 to 0.2 mg, 0.1 to 0.2 mg, 0.9 to 1.2 mg, 0.15 to 1.2 mg, 0.10 to 1.5 mg, or 0.10 to 2 mg of sNAG nanofibers are administered to a subject as a dose to the scalp, hair or both. In some embodiments, 0.5 to 3 mg, 0.5 to 2 mg, 0.5 to 1 mg, 1 to 3 mg, 2 to 3 mg, 0.8 to 1 mg, 0.8 to 2 mg, or 0.8 to 1 mg of sNAG nanofibers are administered to the scalp, hair or both of a subject daily. In a specific embodiment, the composition is sprayed onto the scalp, hair or both.

In a specific embodiment, approximately 100 microliters of a composition described herein is applied multiple times (e.g., 8 to 30 times) to the scalp, hair or both of a subject. In another specific embodiment, approximately 100 microliters of a composition described herein is applied 5 to 30 times, 5 to 25 times, 5 to 20 times, 5 to 15 times, or 5 to 10 times to the scalp, hair or both of a subject. In another specific embodiment, approximately 100 microliters of a composition described herein is applied 10 to 30 times, 10 to 25 times, 10 to 20 times, or 10 to 15 times to the scalp, hair or both of a subject. In another specific embodiment, approximately 100 microliters of a composition described herein is applied 15 to 30 times, 15 to 25 times, or 15 to 20 times to the scalp, hair or both of a subject. In a specific embodiment, the composition is sprayed onto the scalp, hair or both. In certain embodiments, 0.5 to 3 mg, 0.5 to 2 mg, 0.5 to 1 mg, 1 to 3 mg, 2 to 3 mg, 0.8 to 1 mg, 0.8 to 2 mg, or 0.8 to 1 mg of sNAG nanofibers are applied to the scalp, hair or both of a subject daily.

In specific embodiments, approximately 100 microliters of a composition described herein is sprayed multiple times (e.g., 5 to 30, 5 to 25, 5 to 20, 5 to 15, or 5 to 10 times) on the over the entire scalp and hair, and the composition is massaged into the hair and scalp for a period of time (e.g., for approximately 10-15 seconds, approximately 10-30 seconds, approximately 30 to 60 second, approximately 30 to 90 seconds, approximately 1 to 3 minutes, or approximately 1 to 5 minutes). In certain embodiments, 0.5 to 3 mg, 0.5 to 2 mg, 0.5 to 1 mg, 1 to 3 mg, 2 to 3 mg, 0.8 to 1 mg, 0.8 to 2 mg, or 0.8 to 1 mg of sNAG nanofibers are applied to the scalp, hair or both of a subject daily for a period of time (e.g., for about 60 days, about 90 days, about 4 months, about 5 months, about 6 months, about 7 months, about 8 months, about 9 months or longer).

4.7 Combination Therapy

In some embodiments, sNAG nanofibers or a sNAG nanofiber composition described herein are/is administered in conjunction with an anti-bacterial agent, for example an antibiotic. In other embodiments, a composition described herein is not administered in conjunction with an anti-bacterial agent, for example an antibiotic. In some embodiments, sNAG nanofibers or a sNAG nanofiber composition described herein are/is administered in conjunction with an anti-viral agent or anti-fungal agent. In other embodiments, sNAG nanofibers or a sNAG nanofiber composition described herein are/is not administered in conjunction with anti-viral agent or anti-fungal agent.

In some embodiments, the sNAG nanofibers or a sNAG nanofiber composition described herein are/is administered in conjunction with composition that promotes hair growth, hair thickness, hair body, and hair health. In some embodiments, the sNAG nanofibers or a composition thereof are/is administered in conjunction with a penetration enhancer, a hair growth promoter, a circulation promoter, or an anti-inflammatory agent. In certain embodiments, the sNAG nanofibers or a sNAG nanofiber composition described herein are/is administered in conjunction with composition that reduces hair shedding. In specific embodiments, the sNAG nanofibers or a sNAG nanofiber composition described herein are/is administered in conjunction with any one, two or more, or all of the following: Rogaine, Minoxidil, steroids (oral or topical), corticosteroids, anthralin cream, spironolactone, flutamide, ketoconazole, and pyrithione zinc. In some embodiments, the sNAG nanofibers or a sNAG nanofiber composition described herein are/is administered in conjunction with a nutritional supplement. In certain embodiments, the sNAG nanofibers or a sNAG nanofiber composition described herein are/is administered in conjunction with immunotherapy. In some embodiments, the sNAG nanofibers or a sNAG nanofiber composition described herein are/is administered in conjunction with any one, two or more of the following: terbinafine, fluconazole, itraconazole, or griseofulvin. In certain embodiments, sNAG nanofibers or a composition thereof are/is administered in conjunction with one, two, or more, or all of the following ingredients: Methylsulfonylmethane, Copper Tripeptide-1, Curcuma longa Peptides, Caffeine, Biotin, Album (Sandalwood) Wood Extract, Pisum sativum (Pea) Sprout Extract, and Keratinocyte Growth Factor.

In some embodiments, the sNAG nanofibers or sNAG nanofiber compositions described herein are administered before (e.g., 1 minute, 15 minutes, 30 minutes, 1 hour, 2 hours, 3 hours, 6 hours, 12 hours, 24 hours or more before, or any time period in between), simultaneously with, or after (e.g., 1 minute, 15 minutes, 30 minutes, 1 hour, 2 hours, 3 hours, 6 hours, 12 hours, 24 hours or more after, or any time period in between) administration of another therapy or agent.

In some embodiments, the sNAG nanofibers or a sNAG nanofiber composition described herein are/is not administered in conjunction with composition that promotes hair growth, hair thickness, hair body, and hair health. In some embodiments, the sNAG nanofibers or a composition thereof are/is not administered in conjunction with a penetration enhancer, a hair growth promoter, a circulation promoter, or an anti-inflammatory agent. In certain embodiments, the sNAG nanofibers or a sNAG nanofiber composition described herein are/is not administered in conjunction with composition that reduces hair shedding. In specific embodiments, the sNAG nanofibers or a sNAG nanofiber composition described herein are/is not administered in conjunction with any one, two or more, or all of the following: Rogaine, Minoxidil, steroids (oral or topical), corticosteroids, anthralin cream, spironolactone, flutamide, ketoconazole, and pyrithione zinc. In some embodiments, the sNAG nanofibers or a sNAG nanofiber composition described herein are/is not administered in conjunction with a nutritional supplement. In certain embodiments, the sNAG nanofibers or a sNAG nanofiber composition described herein are/is not administered in conjunction with immunotherapy. In some embodiments, the sNAG nanofibers or a sNAG nanofiber composition described herein are/is not administered in conjunction with any one, two or more of the following: terbinafine, fluconazole, itraconazole, or griseofulvin. In certain embodiments, sNAG nanofibers or a composition thereof are/is not administered in conjunction with one, two, or more, or all of the following ingredients: Methylsulfonylmethane, Copper Tripeptide-1, Curcuma longa Peptides, Caffeine, Biotin, Album (Sandalwood) Wood Extract, Pisum sativum (Pea) Sprout Extract, and Keratinocyte Growth Factor.

4.8 Kits

A pack or kit which comprises any of the above-described sNAG compositions is also contemplated. The pack or kit may comprise one or more containers filled with the compositions described herein. The composition is preferably contained within a sealed, waterproof, package which facilitates removal of the composition without contamination. Materials from which containers may be made include aluminum foil, plastic, or another conventional material that is easily. The composition may be packaged in for example, a 25 mL, 50 mL, 75 mL, 100 mL, 125 mL, 150 mL, 175 mL or 200 mL spray bottle. The kit can contain material for a single administration or multiple administrations of the composition. In a preferred embodiment, the kit contains material for multiple administrations of the composition. In a specific embodiment, the composition is a serum. Optionally associated with such kit or pack can be a notice in the form regarding the manufacture, use or sale of a composition described herein for human administration. For example, a kit can comprise a notice regarding instructions for use. In a specific embodiment, the instructions state the following: Use once daily, morning or night, as a leave-in scalp treatment. Apply after shampooing/conditioning or on dry scalp on days when you are not shampooing/conditioning. Part the hair and spray at the roots, as close to the scalp as possible, several times, until the scalp is covered with serum. Thoroughly massage scalp for 25-30 seconds. In some embodiments, the instructions state to use the composition once a day for the first 3 months, then 1-3 times a week for maintenance.

The kits encompassed herein can be used in the above applications and methods.

### 5. EXAMPLES

### 5.1 Example 1: Shortened Fibers of Poly-N-Acetyl Glucosamine

Reduce Hair Shedding

This example demonstrates that effectiveness a composition comprising shortened fibers of poly-N-acetyl glucosamine in reducing the amount of hair shedding by healthy women without causing any adverse reaction.

5.1.1 Objectives of the Study

This study was intended to assess the effect of shortened fibers of poly-N-acetyl glucosamine on hair shedding in healthy women having hair shedding concerns (either seasonal, post pregnancy, due to aging reasons, stress etc.) and to check its skin acceptability, after repeated product application, once a day for 14 consecutive days under normal conditions of use.

5.1.2 Materials and Methods

Test Product: Shortened fibers of poly-N-acetyl glucosamine suspended in water at a concentration 1 mg/mL with caprylyl glycol and phenoxyethanol. The shortened fibers of poly-N-acetyl glucosamine are approximately 70% or more acetylated and have a molecular weight of 70,000 daltons±6,000 daltons and an average fiber length of 1 μm to 5 μm.

5.1.2.1. Subjects

10 women were included in the study that met the inclusion criteria below and did not meet the criteria below under exclusion criteria below.

Inclusion Criteria for the Study:

1. Aged from 18 to 65

### 2. Female

3. With any hair length (short but not shorter than 5 cm, medium or long hair) and shape (straight, curly, wavy)
4. With a phototype (Fitzpatrick): I, II, III or IV
5. With hair shedding concern (either seasonal, post pregnancy, due to aging reasons, stress etc.)
6. With thinning hair and loss of hair volume
7. Showing a hair count of the “Hair Pull Test” of a minimum 15 (last shampoo 2 days before)
8. Agreeing not to take any treatment (oral or topic) able to interfere with the hair growth, diameter or hair fall during the whole study duration
9. Certifying not to take part in another clinical study that could interfere with the current study
10. Capable of following directions and reliable to respect the constraints of the protocol

Exclusion Criteria from the Study:

1. With family or personal history of atopy
2. Who were diagnosed with hair loss conditions (alopecia areata, cyclic alopecia, loose anagen syndrome, acute anagen and trichotillomania)
3. Who were diagnosed with chronic skin allergies
4. Who were receiving any topical or systemic hair loss or hair growth treatments (at the time of or in the last 6 weeks prior to enrollment (Rogaine, Minoxidil, nutritional supplement)
5. With personal history of adverse reactions to the same type of product as the investigational product (hair products)
6. Suffering from dermatological affections on scalp which would be able to interfere with the interpretation of the results.
7. Under treatment, prior to the study, able to interfere with the interpretation of the study results

5.1.2.2. Assessment of the Anti-Shedding Effect

The anti-shedding effect was based on the Hair Pull Test (HPT) performed on 3 areas of the scalp (fronto-temporal, parietal and occipital) by a qualified investigating technician:

at Day 0—before starting product application; and

after Day 14 (Day End).

The anti-shedding effect of the investigational product was assessed by comparing results obtained at Day End to those obtained on Day 0.

5.1.2.3. Hair Pull Test (HPT)

HPT is performed on hair unwashed for 2 days, neither brushed nor combed within 2 hours before examination, 3 areas of the scalp (fronto-temporal, parietal and occipital) are chosen.

The test is based on the concept of ‘gently’ pulling of the hair to bring about shedding of telogen hairs so HPT allows to roughly evaluate the intensity of the hair shedding.

The principle of the HPT consists of slightly pulling about 60 hairs, on 3 delineated scalp areas to score the hair shed. A clump of about 60 hairs per area is taken between the thumb and the forefinger and slightly pulled. This is done by a trained technician.

5.1.2.4. Local Tolerance Assessment

Local tolerance of the product was assessed by a scalp examination by the investigator:

before product application (Day 0); and

after 14 days (Day end).

5.1.2.5. Application of the Investigational Products

The investigational product was applied at home by each subject for a duration of 14 days. The subjects were instructed to apply the product once a day starting with Day 1 until Day 14.

The investigational product was applied in the morning, after regular hair care routine (shampoo and condition the hair as usual). The subjects were instructed to part their hair and spray a generous amount of the product directly on the scalp. The subjects were instructed to do this several times to cover the entire scalp and hair. Following product spraying, the subjects were instructed to massage the product into their hair and scalp for 10-15 seconds then style the hair as usually. After product application on wet hair, leave it in and style normally (blow dry or towel dry).

The subjects were instructed to follow their normal hair care routine (except for day 0 and day 14). The test evaluation was done on hair unwashed for 2 days, neither brushed nor combed within 2 hours before examination.

5.1.3 Results

Assessment of the Anti-Shedding Effect:

As shown in Table 1 below, there was a 51.04% reduction in the amount of hair shedding following 14 daily treatments with shortened fibers of poly-N-acetyl glucosamine. The difference in hair shedding between Day 0 and Day End was statistically significant (Paired t test P<0.001).

TABLE 1

| | | | Std. | | |
|---|---|---|---|---|---|
| | Mean | Median | Deviation | Minimum | Maximum |
| Day 0 (sNAG) | 19.20 | 18.50 | 2.49 | 16.00 | 24.00 |
| Day End | 9.40 | 9.00 | 2.55 | 6.00 | 15.00 |
| (sNAG) | | | | | |
| Difference | −9.80 | | | | |
| % of variation | −51.04% | | | | |

Scalp Examination:

No adverse reactions were observed by the investigator during the study. Subjects reported high levels of satisfaction with the test product with no sensations of discomfort.

### 5.2 Example 2: Shortened Fibers of Poly-N-Acetyl Glucosamine Accelerates Hair Growth

This example demonstrates that a composition comprising shortened fibers of poly-N-acetyl glucosamine accelerates hair regrowth in healthy men.

5.2.1 Objectives of the Study

Efficacy of the test product was measured via Hair Counting.

The study and protocol were carried out under reviewed by an Institutional Review Board. An informed consent was obtained from each volunteer prior to initiating the study describing reasons for the study, possible adverse effects, associated risks and potential benefits of the treatment.

5.2.2 Materials & Methods

5.2.2.1. Test Product

Shortened fibers of poly-N-acetyl glucosamine suspended in water at a concentration 1 mg/mL with caprylyl glycol and phenoxyethanol. The shortened fibers of poly-N-acetyl glucosamine are approximately 70% or more acetylated and have a molecular weight of 70,000 daltons±6,000 daltons and an average fiber length of 1 μm to 5 μm.

5.2.2.2. Subjects

Three male subjects were included in the study that met the inclusion criteria below and did not meet the criteria under the exclusion criteria below.

Inclusion Criteria:

1. Three men between 20 and 65 years old.
2. Complete a preliminary medical history and screening document
3. Individuals, who will read, understood and signed an informed consent document as required by CFR Title 21, Part 50, Subpart B regulations.
4. Individuals in general good health and free of any health problems, including neurological, dermatological, or systemic disorder that in the opinion of the Study Director would make study participation inappropriate.
5. Individuals able to cooperate with the Investigator and research staff, willing to have the test material(s) applied according to the protocol, and complete the full course of study.
6. Individuals experiencing patterned baldness (not complicated with other crucial hair disorders such as alopecia areata-cyclic alopecia, loose anagen syndrome, acute anagen or telogen effluvium and trichotillomania etc.) as confirmed by the Study Director.
7. Individuals who agreed to maintain the same hair style, hair length and hair colour during the entire study.

Exclusion Criteria:

1. Individuals who are under the care of a physician being treated for specific condition that may interfere with the study design at the discretion of the Study Director.
2. Individuals currently taking medication that may mask or interfere with the test results.
3. Individuals diagnosed with chronic skin allergies.
4. Individuals suffering from hypothyroidism or receiving a thyroid hormone treatment in the last 6 weeks prior to enrolment.
5. Subjects with a history of any form of skin cancer, melanoma, lupus, psoriasis, connective tissue disease, diabetes, or any disease that would increase the risk associated with study participation.
6. Individuals who have experienced irritation or sensitivity to topical products.
7. Individuals with known allergies, scalp inflammation or skin conditions, which would interfere with the study at the discretion of the Study Director.
8. Individuals receiving any hair loss treatments currently or in the last 6 weeks prior to enrolment.
9. Individuals participating in any clinical research study at another facility or with a doctor's office at the commencement and duration of the study.

5.2.2.3. Assessment of Hair Regrowth

Three healthy male subjects between the ages of 30 to 65 were inducted into this study.

Hair Counting was conducted at the baseline (before product application) and again after 2 weeks. Hair Counting was done using PhotoGrammetrix™ Image Analysis readings collected at Baseline and Day 14. The exact same area of the scalp was captured at Baseline and again on Day 14 and the hairs were counted digitally through the computer.

During the baseline qualification, each panelist was evaluated by a Trained Clinical Evaluator. The scalp of each panelist was examined to rule out the presence of any confounding scalp conditions. Only the individuals experiencing patterned baldness at Hamilton-Norwood scale—1-2 to 11-2 were inducted into the study.

The scalp was shaved to ensure equal length of hair shaft in all subjects. Panelists received verbal and written instructions regarding product use and study restrictions. Subjects were required to use the test product on the right side of their head only as a part of their daily routine. The left side of the head was left as an untreated control.

The test product was applied at night, before bed, on dry hair directly on the right side of the scalp, the treated area was massaged with the hand/fingers for 10-15 seconds. The next morning, the subjects were instructed to use normal hair care routine including regular hair care products.

The subjects were instructed to continue to apply the product according to instructions every day for 14 days.

Study participants were asked to return to the test facility after week 2 of product use.

5.2.2.4. Statistical Source Data

The source data included Hair Counting readings collected prior to application and after 2 weeks of use of the test product. The data used in the statistical analysis reflect changes from baseline. All readings were totaled and reported as average scores. The obtained data was quoted as % differences from baseline at each of the previously described time points. A within group comparison of baseline measurements with post-treatment measurements was analyzed using a (two-tailed, paired) t-test, (p<0.05).

5.2.2.5. Local Tolerance and Global Assessment

A scalp examination was conducted by a trained investigator at baseline and after 2 weeks of use of the test product.

5.2.3 Results

There was improvement in the overall condition of the hair following treatment with shortened fibers of poly-N-acetyl glucosamine. As shown in Table 2 below, the Hair Counting analysis showed the shortened fibers of poly-N-acetyl glucosamine accelerated hair regrowth. No adverse reactions were observed by the investigator when examining the scalp during the study.

TABLE 2
Hair Counting Analysis

| | Right Side | | | Left Side | | |
|---|---|---|---|---|---|---|
| | SNAG | | | Untreated | | |
| | | | Individual % | | | Individual % |
| Panelist ID No.: | Baseline | Day 14 | Difference | Baseline | Day 14 | Difference |
| 62 2294 | 80352.00 | 482616.00 | 500.63% | 84511.00 | 352679.00 | 317.32% |
| 50 9089 | 117122.00 | 666444.00 | 469.02% | 82304.00 | 452618.00 | 449.93% |
| 74 3869 | 143004.00 | 925004.00 | 546.84% | 124339.00 | 645435.00 | 419.09% |
| Average: | 113492.67 | 691354.67 | | 97051.33 | 483577.33 | |
| Average (Adjusted): | 105272.00 | 641277.46 | | 105272.00 | 524538.42 | |

| | | |
|---|---|---|
| % Difference | 509.16% | 398.27% |

### 5.3 Example 3: Shortened Fibers of Poly-N-Acetyl Glucosamine Improve the Condition of Hair

This example demonstrates that a composition comprising shortened fibers of poly-N-acetyl glucosamine is effective in improving the overall condition of the hair and accelerates hair distal length and thickness (diameter) growth.

5.3.1 Objectives of the Study

Efficacy of the test product was evaluated by measuring distal length and diameter (thickness) at baseline treatment.

The study and protocol were carried out under review by an Institutional Review Board. An informed consent was obtained from each volunteer prior to initiating the study describing reasons for the study, possible adverse effects, associated risks and potential benefits of the treatment.

5.3.2 Materials and Methods:

5.3.2.1. Test Product

Shortened fibers of poly-N-acetyl glucosamine suspended in water at a concentration 1 mg/mL with caprylyl glycol and phenoxyethanol. The shortened fibers of poly-N-acetyl glucosamine are approximately 70% or more acetylated and have a molecular weight of 70,000 daltons±6,000 daltons and an average fiber length of 1 μm to 5 μm.

5.3.2.2. Subjects

Five female subjects were included in the study that met the inclusion criteria below and did not meet the criteria under the exclusion criteria below.

Inclusion Criteria:

1. Five healthy female subjects between the ages of 20 and 65 years old
2. Complete a preliminary medical history and screening document
3. Individuals, who will read, understood and signed an informed consent document as required by CFR Title 21, Part 50, Subpart B regulations.
4. Individuals in general good health and free of any health problems, including neurological, dermatological, or systemic disorder that in the opinion of the Study Director would make study participation inappropriate.
5. Individuals able to cooperate with the Investigator and research staff, willing to have the test material(s) applied according to the protocol, and complete the full course of study.
6. Individuals experiencing patterned baldness (not complicated with other crucial hair disorders such as alopecia areata—cyclic alopecia, loose anagen syndrome, acute anagen or telogen effluvium and trichotillomania etc.) as confirmed by the Study Director.
7. Individuals who agreed to maintain the same hair style, hair length and hair colour during the entire study.

Exclusion Criteria:

1. Individuals who are under the care of a physician being treated for specific condition that may interfere with the study design at the discretion of the Study Director.
2. Individuals currently taking medication that may mask or interfere with the test results.
3. Individuals diagnosed with chronic skin allergies.
4. Individuals suffering from hypothyroidism or receiving a thyroid hormone treatment in the last 6 weeks prior to enrolment.
5. Subjects with a history of any form of skin cancer, melanoma, lupus, psoriasis, connective tissue disease, diabetes, or any disease that would increase the risk associated with study participation.
6. Individuals who have experienced irritation or sensitivity to topical products.
7. Individuals with known allergies, scalp inflammation or skin conditions, which would interfere with the study at the discretion of the Study Director.
8. Individuals receiving any hair loss treatments currently or in the last 6 weeks prior to enrolment.
9. Individuals participating in any clinical research study at another facility or with a doctor's office at the commencement and duration of the study.

5.3.2.3. Assessment of Hair Regrowth

Five healthy female subjects between the ages of 20 and 65 were inducted into this study. During the baseline qualification each panelist was evaluated by a Trained Clinical Evaluator. The scalp of each panelist was examined to rule out the presence of any confounding scalp conditions.

Subjects were required to use the test product on the right side of their head only as a part of their daily routine. The left side of the head was left as an untreated control.

Hair measurements were conducted at the baseline (before product application) and again after 2 weeks. For each of the five subjects, five hair shafts were plucked at random from the treated (right) and five from the untreated (left) side. The hair shafts were measured for distal length (cm) and diameter (mm, optical micrometry).

The subjects received verbal and written instructions regarding product use and study restrictions. Subjects were required to use the test product on the right side of their head only as a part of their daily routine. The left side of the head was left as an untreated control.

The subjects were instructed to apply the test product in the morning, after their regular hair care routine (shampoo and condition the hair as usual). The subjects were instructed to part their hair and spray a generous amount of the product directly on the right side of their scalp and to do this several times to cover the entire scalp and hair. Following product spraying, the subjects were instructed to massage the product into the hair and scalp for 10-15 seconds and then style their hair as usual. After product application on wet hair, the subjects were instructed to leave it in and style normally (blow dry or towel dry). The subjects were instructed to continue to apply the product according to instructions every day for 14 days.

Study participants were asked to return to the test facility after week 2 of product use.

5.3.2.4. Local Tolerance

A scalp examination was conducted by a trained investigator at baseline and after 2 weeks of use of the test product.

5.3.3 Results

Shortened fibers of poly-N-acetyl glucosamine were effective in improving the overall condition of the hair. As shown in Tables 3 and 4 below, the shortened fibers of poly-N-acetyl glucosamine accelerated hair distal length and thickness (diameter) growth. In addition, no adverse reactions were observed by the investigator during the study.

TABLE 3
Hair Diameter (in mm, optical micrometry)
Average of five hair shafts per subject

| | Treatment Area (right side) | | Control Area (left side) | |
|---|---|---|---|---|
| Subject | Baseline | 14 Days | Baseline | 14 Days |
| 64 7381 | 0.03530 | 0.03593 | 0.03520 | 0.03553 |
| 68 4439 | 0.03524 | 0.03566 | 0.03535 | 0.03595 |
| 60 8960 | 0.03502 | 0.03568 | 0.03500 | 0.03545 |
| 58 7412 | 0.03542 | 0.03597 | 0.03548 | 0.03593 |
| 80 7739 | 0.03494 | 0.03582 | 0.03511 | 0.03581 |
| | 0.03518 | 0.03581 | 0.03523 | 0.03573 |
| | | 1.78% | | 1.44% |

TABLE 4
Hair Distal Length (cm)
Average of five hair shafts per subject

| | Treatment Area (right side) | | Control Area (left side) | |
|---|---|---|---|---|
| Subject | Baseline | 14 Days | Baseline | 14 Days |
| 64 7381 | 31.28 | 38.32 | 39.00 | 37.02 |
| 68 4439 | 29.94 | 35.92 | 28.26 | 32.18 |
| 60 8960 | 11.24 | 13.16 | 17.54 | 15.02 |
| 58 7412 | 27.34 | 28.66 | 31.74 | 30.10 |
| 80 7739 | 33.24 | 34.82 | 36.68 | 37.80 |
| Average | 26.61 | 30.18 | 30.64 | 30.42 |
| | | 13.41% | | −0.72% |

### 5.4 Example 4: Effect of Chitosan and Glucosamine on Hair Shedding

This example demonstrates that glucosamine is more effective than chitosan in reducing hair shedding by healthy women.

5.4.1 Objectives of the Study

This study was intended to assess the effect of Chitosan and Glucosamine on hair shedding in healthy women having hair shedding concerns (either seasonal, post pregnancy, due to aging reasons, stress etc.) and to check its skin acceptability, after repeated product application, once a day for 14 consecutive days under normal conditions of use.

5.4.2 Materials and Methods

5.4.2.1. Test Products

Chitosan (Carbomer, SKU: 4-00559) suspended in water at a concentration of 1 mg/mL. The chitosan is reported to have a granule size <0.2 mm, an ash <1.0%, a degree of deacetylation >80%, and a high viscosity (1% 6,000 mPas in aqueous acetic acid).

Glucosamine (D(+)-glucosamine hydrochloride; Sigma) suspended in water at a concentration of 1 mg/mL.

5.4.2.2. Subjects

12 female subjects (6 in each group) were included in the study that met the inclusion criteria below and did not meet the criteria under the exclusion criteria below.

Inclusion Criteria:

1. Aged from 18 to 65

### 2. Female

3. With any hair length (short but not shorter than 5 cm, medium or long hair) and shape (straight, curly, wavy)
4. With a phototype (Fitzpatrick): I, II, III or IV
5. With hair shedding concern (either seasonal, post pregnancy, due to aging reasons, stress etc.)
6. With thinning hair and loss of hair volume
7. Showing a hair count of the “Hair Pull Test” of a minimum 15 (last shampoo 2 days before)
8. Agreeing not to take any treatment (oral or topic) able to interfere with the hair growth, diameter or hair fall during the whole study duration
9. Certifying not to take part in another clinical study that could interfere with the current study
10. Capable of following directions and reliable to respect the constraints of the protocol

Exclusion Criteria:

1. With family or personal history of atopy
2. Who were diagnosed with hair loss conditions (alopecia areata, cyclic alopecia, loose anagen syndrome, acute anagen and trichotillomania)
3. Who were diagnosed with chronic skin allergies
4. Who were receiving any topical or systemic hair loss or hair growth treatments (at the time of or in the last 6 weeks prior to enrollment (Rogaine, Minoxidil, nutritional supplement)
5. With personal history of adverse reactions to the same type of product as the investigational product (hair products)
6. Suffering from dermatological affections on scalp which would be able to interfere with the interpretation of the results.
7. Under treatment, prior to the study, able to interfere with the interpretation of the study results

5.4.2.3. Assessment of the Anti-Shedding Effect

The anti-shedding effect was based on the Hair Pull Test (HPT) performed on 3 areas of the scalp (fronto-temporal, parietal and occipital) by a qualified investigating technician:

at Day 0—before starting product application; and

after Day 14 (Day End)

The anti-shedding effect of the investigational product was assessed by comparing results obtained at Day End to those obtained on Day 0.

5.4.2.4. Hair Pull Test (HPT)

HPT was performed on hair unwashed for 2 days, neither brushed nor combed within 2 hours before examination; 3 areas of the scalp (fronto-temporal, parietal and occipital) are chosen for examination.

The test is based on the concept of ‘gently’ pulling of the hair to bring about shedding of telogen hairs so HPT allows to roughly evaluate the intensity of the hair shedding.

The principle of the HPT consists of slightly pulling about 60 hairs, on 3 delineated scalp areas to score the hair shed. A clump of about 60 hairs per area is taken between the thumb and the forefinger and slightly pulled. This is done by a trained technician.

5.4.2.5. Local Tolerance Assessment

Local tolerance of the product was assessed by a scalp examination by the investigator or technician:

before product application (Day 0); and

after 14 days (Day end).

5.4.2.6. Application of the Investigational Products

The investigational product was applied at home by each subject for a duration of 14 days. The subjects were instructed to apply the product once a day starting with Day 1 until Day 14. Six women applied chitosan to their scalp and six women applied glucosamine to their scalp.

The investigational product was applied in the morning, after regular hair care routine (shampoo and condition the hair as usual). The subjects were instructed to part their hair and spray a generous amount of the product directly on the scalp. The subjects were instructed to do this several times to cover the entire scalp and hair. Following product spraying, the subjects were instructed to massage the product into their hair and scalp for 10-15 seconds then style the hair as usually. After product application on wet hair, leave it in and style normally (blow dry or towel dry).

The subjects were instructed to follow their normal hair care routine (except for day 0 and day 14). The test evaluation was done on hair unwashed for 2 days, neither brushed nor combed within 2 hours before examination.

5.4.3 Results

5.4.3.1. Assessment of the Anti-Shedding Effect and Scalp Examination—Chitosan

As shown in Table 5 below, there was a 31.86% reduction in the amount of hair shedding following 14 daily treatments with Chitosan. The difference in hair shedding between Day 0 and Day End was statistically significant (Paired t test P<0.001). No adverse reactions were observed by the investigator during the study.

TABLE 5

| | | | Std. | | |
|---|---|---|---|---|---|
| | Mean | Median | Deviation | Minimum | Maximum |
| Day 0 | 18.83 | 18.50 | 1.94 | 17.00 | 22.00 |
| Day 14 | 12.83 | 13.50 | 2.48 | 10.00 | 15.00 |
| Difference | −6.00 | | | | |
| % of | −31.86% | | | | |
| Variation | | | | | |

5.4.3.2. Assessment of the Anti-Shedding Effect and Scalp Examination—Glucosamine

As shown in Table 6 below, there was a 38.4% reduction in the amount of hair shedding following 14 daily treatments with glucosamine. The difference in hair shedding between Day 0 and Day End was statistically significant (Paired t test P=0.001). No adverse reactions were observed by the investigator during the study.

TABLE 6

| | | | Std. | | |
|---|---|---|---|---|---|
| | Mean | Median | Deviation | Minimum | Maximum |
| Day 0 | 18.67 | 18.50 | 1.63 | 17.00 | 21.00 |
| Day End | 11.50 | 10.50 | 2.07 | 10.00 | 15.00 |
| Difference | −7.17 | | | | |
| % of | −38.40% | | | | |
| Variation | | | | | |

5.4.4 Conclusions

There was a reduction of 31.86% reduction in the amount of hair shedding following 14 daily treatments with chitosan, whereas there was a reduction of 38.4% reduction in the amount of hair shedding following 14 daily treatments with glucosamine. The glucosamine yielded a greater reduction in hair shedding than the chitosan.

### 5.5 Example 5: Treatment of Mild to Moderate Scalp Psoriasis

This example demonstrates the successful use of a composition comprising shortened fibers of poly-N-acetyl glucosamine for the treatment of mild to moderate scalp psoriasis.

5.5.1 Background

Maintaining a healthy scalp can prevent many causes of pattern hair loss. Scalp psoriasis causes inflammation and hair loss and treatment options are limited particularly for mild/moderate disease.

5.5.2 Objective of the Study

The aim of this study was to evaluate the efficacy of shortened fibers of poly-N-acetyl glucosamine (concentration 1 mg/mL suspended in water) in the treatment of mild to moderate scalp psoriasis. The shortened fibers of poly-N-acetyl glucosamine are approximately 70% or more acetylated and have a molecular weight of 70,000 daltons±6,000 daltons and an average fiber length of 1 μm to 5 μm.

5.5.3 Materials and Methods:

This was a 14-day randomized placebo-controlled study with 9 patients (5 female/4 male; ages 22-60) presenting with mild to moderate scalp psoriasis, complaining of itching and burning.

Each subject was given either the Active Ingredient (shortened fibers of poly-N-acetyl glucosamine) or Placebo (USP Water) and instructed to apply on the affected scalp daily. At the end of the study subjects receiving Placebo were crossed-over to Active Ingredient.

Clinical evaluation at Day 0, 7 and 14 included 1) Dermoscopy (twisted vessels involving >50% of field at 20×, 5 level scale), 2) Itching/Burning sensation, 1 to 10 subjective scale and 3) degree of erythema/scaling and assessment of PSSI.

5.5.4 Results

A statistically significant reduction in clinical and dermoscopic signs of inflammation (twisted capillary 57%, scales 78%, erythema 69%) was observed between Active Ingredient and Placebo Subjects. Reductions in itching and burning were also reported (itching 78%, burning 82%). Patients crossing from Placebo to Active Ingredient showed similar improvements. The shortened fibers of poly-N-acetyl glucosamine were very well tolerated and none of the subjects complained of side effects.

5.5.5 Conclusions

Our preliminary data show that shortened fibers of poly-N-acetyl glucosamine have important anti-inflammatory action and can be an effective topical treatment option in patients with mild to moderate scalp psoriasis. The subjects tolerated the treatment very well and reported a healthy rejuvenated scalp.

### 5.6 Example 7: Effectiveness of Poly-N-Acetyl-Glucosamine for the Treatment of Hair Loss Associated with Scalp Inflammation

This example demonstrates the effectiveness of a composition comprising shortened fibers of poly-N-acetyl-glucosamine for the treatment of hair loss associated with scalp inflammation.

5.6.1 Background

Oxidative stress and scalp inflammation cause telogen effluvium, affect the health of the follicle and could trigger androgenetic alopecia. Scalp inflammation is commonly associated with scalp psoriasis or seborrheic dermatitis. Patients are often symptomatic and complain of itching and burning. Trichoscopy has been used to diagnose and grade severity of inflammatory changes in these conditions. In scalp psoriasis trichoscopy shows red dots and twisted/glomerular capillary loops. In seborrheic dermatitis it shows an increased number of arborizing vessels. A severity scale was developed to assess inflammation as a % of scalp showing glomerular, arborizing vessels or scales under 20× magnification (Grade 5:100%; Grade 4: 75%; Grade 3: 50%; Grade 2: 25%; Grade 1: between 10% and 25%, Grade 0: ≤10%).

5.6.2 Objective of the Study

The aim of this study was to evaluate the efficacy of shortened fibers of poly-N-acetyl glucosamine (1 mg/mL suspended in water) in the treatment of scalp inflammation due to mild/moderate scalp psoriasis or seborrheic dermatitis. The shortened fibers of poly-N-acetyl glucosamine are approximately 70% or more acetylated and have a molecular weight of 70,000 daltons±6,000 daltons and an average fiber length of 1 μm to 5 μm.

5.6.3 Methods

The evaluation involved a 14-day randomized placebo-controlled study in 20 Subjects (11 females/9 males, ages 18-60) presenting with mild to moderate scalp psoriasis or seborrheic dermatitis complaining of itching and burning. Subjects with scores ≥7 in the itching/burning scale (1 to 10) and dermoscopic scalp inflammation score ≥of 2 were given the opportunity to participate in the study. Each subject was randomly assigned to either the Active Ingredient (shortened fibers of poly-N-acetyl glucosamine) or Placebo (USP Water) and instructed to apply on the affected scalp daily. Clinical evaluation at Day 0-7 and 14 included: 1) Dermoscopy (twisted/glomerular or arborizing vessels involving >50% of field at 20× magnification, 5 level scale), 2) itching/burning sensation 1 to 10 subjective scale, 3) degree of erythema/scaling and 4) tolerability and patient's satisfaction.

5.6.4 Results

At day 14, subjects in the Active Ingredient had a statistically significant reduction in clinical and dermoscopic signs of inflammation (twisted capillary 58%, scales 64%, erythema 69%) compared to Placebo subjects (twisted capillary 0%, scales 0%, erythema −10%). Significant reductions in itching and burning were also reported (Active Ingredient: itching 70%, burning 78%. Placebo: itching 3%, burning 10%). The shortened fibers of poly-N-acetyl glucosamine were very well tolerated and none of the subjects complained of side effects.

5.6.5 Conclusions

Our preliminary data show that topical shortened fibers of poly-N-acetyl glucosamine have important anti-inflammatory action and can be an effective treatment option in patients with hair loss associated with scalp inflammation. The subjects tolerated the treatment very well and reported a healthy rejuvenated scalp.

### 6. INCORPORATION BY REFERENCE

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for reducing hair shedding, the method comprising applying a composition comprising shortened fibers of poly-N-acetylglucosamine (“sNAG nanofibers”) to the scalp or the scalp and hair of a subject and leaving the composition on the scalp or the scalp and hair for a period of time, wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides.

2. A method for improving hair growth, the method comprising applying a composition comprising shortened fibers of poly-N-acetylglucosamine (“sNAG nanofibers”) to the scalp or the scalp and hair of a subject and leaving the composition on the scalp or the scalp and hair for a period of time, wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides.

3. A method for promoting healthier hair, the method comprising applying a composition comprising shortened fibers of poly-N-acetylglucosamine (“sNAG nanofibers”) to the scalp or the scalp and hair of a subject and leaving the composition on the scalp or the scalp and hair for a period of time, wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides.

4. The method of claim 3, wherein the healthier hair promoted has one, two, or more, or all of the following characteristics: thicker hair, hair with improved texture, hair with greater volume, and softer hair.

5. A method for strengthening hair follicles, the method comprising applying a composition comprising shortened fibers of poly-N-acetylglucosamine (“sNAG nanofibers”) to the scalp or the scalp and hair of a subject and leaving the composition on the scalp or the scalp and hair for a period of time, wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides.

6. A method for thickening the diameter of existing hair fiber, the method comprising applying a composition comprising shortened fibers of poly-N-acetylglucosamine (“sNAG nanofibers”) to the scalp or the scalp and hair of a subject and leaving the composition on the scalp or the scalp and hair for a period of time, wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides.

7. A method for improving hair growth and thickening the diameter of existing hair fiber, the method comprising applying a composition comprising shortened fibers of poly-N-acetylglucosamine (“sNAG nanofibers”) to the scalp or the scalp and hair of a subject and leaving the composition on the scalp or the scalp and hair for a period of time, wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides.

8. A method for treating hair loss, the method comprising applying a composition comprising shortened fibers of poly-N-acetylglucosamine (“sNAG nanofibers”) to the scalp or the scalp and hair of a subject and leaving the composition on the scalp or the scalp and hair for a period of time, wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides.

9. The method of any one of claims 1 to 8, wherein the composition is applied to the scalp or the scalp and hair after shampooing or conditioning the hair.

10. The method of any one of claims 1 to 9, wherein the composition is applied to dry hair.

11. The method of any one of claims 1 to 9, wherein the composition is applied to wet hair.

12. The method of any one of claims 1 to 11, wherein the composition is sprayed onto the scalp or the scalp and hair.

13. The method of any one of claims 1 to 12, wherein approximately 100 microliters of the composition is sprayed multiple times on the entire scalp or the entire scalp and hair

14. The method of claim 13, wherein the composition is sprayed 8 to 30 times.

15. The method of any one of claims 1 to 14, wherein the composition is applied once daily.

16. The method of any one of claims 1 to 15, wherein the composition is applied once daily for a period of approximately 1 to approximately 3 months.

17. The method of any one of claims 1 to 16, wherein the composition is massaged into the scalp or the scalp and hair.

18. The method of claim 17, wherein the composition is massaged into the scalp or the scalp and hair for approximately 10 to 60 seconds.

19. The method of any one of claims 1 to 18, wherein the composition comprises water.

20. The method of claim 19, wherein the composition comprises sNAG nanofibers at a concentration of approximately 1 mg/mL.

21. The method of any one of claims 1 to 20, wherein the composition further comprises phenoxyethanol, caprylyl glycol, and sodium hydroxide.

22. The method of any one claims 1 to 21, wherein the composition further comprises glucosamine.

23. The method of any one of claims 1 to 22, wherein the composition does not comprise an additional ingredient.

24. The method of any one of claims 1 to 23, wherein the subject does not have a viral infection of the scalp or a condition affecting the scalp caused by a viral infection.

25. The method of any one of claims 1 to 24, wherein the subject does not have a bacterial infection of the scalp or a condition affecting the scalp caused by a bacterial infection.

26. The method of any one of claims 1 to 25, wherein the subject does not have a fungal infection of the scalp or a condition affecting the scalp caused by a fungal infection.

27. The method of any one of claims 1 to 26, wherein the subject does not have psoriasis.

28. The method of claim 27, wherein the subject does not have psoriasis affecting the scalp.

29. The method of any one of claims 1 to 28, wherein the subject does not have dermatitis.

30. The method of claim 29, wherein the subject does not have dermatitis affecting the scalp.

31. The method of any one of claims 1 to 30, wherein the subject does not have a wound on the scalp.

32. The method of any one of claims 1 to 31, wherein more than 50% of the sNAG nanofibers are between about 2 to 10 μm in length.

33. The method of any one of claims 1 to 32, wherein the sNAG nanofibers have an average length of between about 1 to about 5 μm in length.

34. The method of any one of claims 1 to 34, wherein 100% of the sNAG nanofibers are between about 1 to 15 μm in length.

35. The method of any one of claims 1 to 34, wherein the length of the sNAG nanofibers is measured by scanning electron microscopy.

36. The method of any one of claims 1 to 35, wherein at least 80%, at least 85%, at least 90%, at least 95%, or at least 99% of the monosaccharides are N-acetylglucosamine monosaccharides

37. The method of any one of claims 1 to 36, wherein the sNAG nanofibers were produced by gamma irradiation of poly-N-acetylglucosamine fibers, and wherein the poly-N-acetylglucosamine fibers were irradiated in the form of dried fibers at 500-2,000 kgy, or the poly-3-N-acetylglucosamine fibers were irradiated in the form of wet fibers at 100-500 kgy.

38. The method of any one of claims 1 to 37, wherein the poly-N-acetylglucosamine fibers have a β-1→4 configuration.

39. The method of any one of claims 1 to 38, wherein the sNAG nanofibers were produced from a microalgae poly-N-acetylglucosamine.

40. The method of any one of claims 1 to 39, wherein the composition is a serum, suspension or a gel.

41. The method of any one of claims 1 to 40, wherein the composition is not administered in conjunction with another therapy or agent.

42. The method of any one of claims 1 to 41, wherein the subject is a human subject.

43. The method of claim 42, wherein the human subject is a human adult.

44. The method of claim 42, wherein the human subject is an elderly human.

45. The method of any one of claims 1 to 44, wherein the period of time is at least 3 hours, 6 hours, 12 hours, 18 hours or 24 hours.

46. The method of any one of claims 1 to 44, wherein the period of time is 3-6 hours, 6-9 hours, 6-12 hours, 9-12 hours, 9 to 18 hours, 12 to 18 hours, 12 to 24 hours, or 18 to 24 hours.

47. The method of any one of claims 1 to 44, wherein the period of time is until the subject washes his or her hair.

48. The method of any one of claims 1 to 47, wherein 0.5 to 3 mg of the sNAG nanofibers are applied to the scalp or the scalp and hair of the subject daily.
